(19) United States
(12) Patent Application Publication
(10) Pub. No.: US 2004/0072185 A1
(43) Pub. Date: Apr. 15, 2004

(54) Cellular genes involved in oncogenesis, products of said genes and their diagnostic and therapeutic uses

(76) Inventors: Patrizia Paterlini-Brechot, Paris (FR); Christian Brechot, Paris (FR)

Correspondence Address: LARSON & TAYLOR, PLC, 1199 NORTH FAIRFAX STREET, SUITE 900, ALEXANDRIA, VA, 22314, US

(21) Appl. No.: 10/380,374

(22) PCT Filed: Jul. 31, 2001
(86) PCT No.: PCT/FR01/02509

(30) Foreign Application Priority Data
Sep. 15, 2000 (FR) 0011826
May 23, 2001 (US) 60292920

(51) Int. Cl. C12Q 1/68; G01N 33/574; C07H 21/04; C07K 14/47; C07K 16/30

(52) U.S. Cl. 435/6; 435/69.1; 435/320.1; 435/325; 435/7.23; 530/350; 530/388.8; 536/23.2

(57) ABSTRACT

The invention concerns the identification of cellular genes involved in oncogenesis, by insertional mutagenesis of the hepatitis B virus. Besides novel genes (including a gene of the MCM family), the invention further concerns the involvement of TRAP-150, SERCA-1, hTERT, CCT7, NMP 84p, TRKB, TRUP, ST3GalVI, and IP₃R₁ genes in cancerology.

The invention relates to the identification of cellular genes involved in oncogenesis, and in particular liable to play a role in hepatic carcinogenesis.

Chronic infection with the hepatitis B virus (HBV), a virus of the Hepadna family, is a major etiological factor in hepatocellular carcinoma (HCC), the most common histological form of primary liver cancer. In the last few years, it has been clearly shown that HBV is involved in the process of hepatic carcinogenesis via three synergistic mechanisms:

a) the immune response to the viral antigens is the basis of a chronic hepatic inflammation which can progress to cirrhosis. Cirrhosis, characterized by hepatocyte regeneration and hepatic fibrosis, is a pretumor pathological condition which progresses to HCC with a frequency of 5% per year;

b) the viral genome determines the expression of viral proteins (X, truncated PreS2/S protein) which can directly modulate cellular signal transduction pathways and, via this, cell proliferation and viability;

c) the viral genome integrates into the cellular genome in virtually all HBV-related HCC carcinomas. This integration has been shown to induce chromosomal instability and, sometimes, to cis-activate (cis-activation or insertional mutagenesis) cellular genes located in the proximity of the integration site (Paterlini et al, 1994).

The conventional cloning strategies used for about twenty years have not made it possible to identify common sites of integration of HBV into the cellular DNA of HCC carcinomas. Genes close to the sites of integration of the virus have been found in only five tumors (Pineau et al, 1996; Zhang et al, 1992; Dejean et al, 1986; Wang et al, 1990; Tsuei et al, 1994), the total number of tumors studied remaining small. In only two cases, namely the integration of the RAR-E gene and that of the cyclin A2 gene, complementary analyses have been carried out to demonstrate the transforming effect of the integration of HBV. Despite this, the general conclusion of these studies was that the insertional mutagenesis relating to HBV was an anecdotal event in HCC.

Taking advantage of a PCR technique using primers specific for Alu sequences (Minami et al, 1995), the authors of the present invention have now succeeded in screening 45 HCC tumors positive for HBV and in isolating, among said tumors, 21 sites of integration of HBV into the cellular genome, from 18 tumors. 13 cellular genomic sequences flanking these integration sites have thus been identified. 11 of these sequences have indicated the existence of genes of interest in proximity to the sites of integration. These genes correspond either to genes unknown to date, or to genes which are known but the involvement of which in oncogenesis had not been reported or confirmed. They are in particular the sequences of the following known genes:

the TRAP 150 gene considered to encode a protein of a nuclear receptor co-activator complex (protein referred to as “Thyroid Hormone Receptor Associated Protein”) (Ito et al., 1999);

the hTERT gene which encodes human telomerase, which catalyzes the synthesis of the telomeric DNA of chromosomes (Urquidi et al., 2000);

the SERCA 1 gene, for Sarco/Endoplasmic Reticulum Calcium ATPase, which encodes a calcium pump which transfers calcium from the cytosol to the endoplasmic reticulum (Chami et al., 2000);

the IP₃R₁ gene (inositol 1,4,5-triphosphate receptor type 1) which encodes an intracellular calcium channel located in the endoplasmic reticulum membrane, and which controls calcium release from this compartment in the direction of the cytosol, in response to signals induced by tyrosine kinase receptors and G protein-coupled plasma membrane receptors.

The nucleic acid sequences identified are given in the attached sequence listing. In this listing, SEQ ID No. 1 is the cellular nucleotide sequence close to a site of integration of HBV into the tumor referred to as FR7 by the inventors, the sequence SEQ ID No. 2 being the transcribed portion of this sequence.

The sequences SEQ ID No. 3 to SEQ ID No. 10 are the nucleotide sequences present as unique sequences in the cellular genome, which are in proximity to the sites of integration of HBV, respectively into the tumors referred to as 54T, 83T, 95T, FR2, FR3, SA1, SA2, and GR2S.

The sequence SEQ ID No. 11 represents the cellular sequence found in proximity to the site of insertion of HBV into the tumor referred to as 77T by the inventors (cellular sequence which is found in the sequence of the Genbank genomic clone AL035461).

The sequences SEQ ID Nos. 1, 3 to 11, 20 and 23-24 correspond to the 13 cellular sequences flanking the sites of integration of HBV into the genome.

The sequence SEQ ID No. 12 is the cDNA sequence isolated from a normal liver by the inventors, identified by them as encoding a new protein which is a member of the MCM (for “Minichromosome maintenance”, Bik K. Tye, 1999) proteins, and which is referred to as MCM8. The corresponding amino acid sequence is represented by SEQ ID No. 13.

The nucleotide sequences SEQ ID Nos. 14, 16 and 18 are sequences also transcribed from the MCM8 gene, subsequent to three different splicings. The sequences SEQ ID Nos. 15, 17 and 19 represent the amino acid sequences respectively encoded by these nucleotide sequences.

The sequence SEQ ID No. 20 represents the cellular sequence found in proximity to the site of insertion of HBV into the tumor referred to as 100T by the inventors (corresponding to the TRAP 150 gene), the sequence SEQ ID No. 21 being the cDNA sequence of the TRAP 150 gene, and the sequence SEQ ID No. 22 being the corresponding amino acid sequence.

The sequences SEQ ID Nos. 23 and 24 represent the cellular sequences found in proximity to the sites of integration of HBV into the tumors referred to as 83T and 86T respectively, corresponding to hTERT and hSERCA1 genes.

The sequence SEQ ID No. 25 represents the sequence of the complete SERCA 1 gene cDNA and the sequence SEQ ID No. 26 represents the corresponding amino acid sequence.

The sequences SEQ ID Nos. 27 and 29 are sequences transcribed from the SERCA1 gene, referred to respectively as S1T+4 and S1T-4, produced by splicing of exon 11 and alternative splicing of exon 4 (the S1T-4 form exhibiting splicing of exon 4 and exon 11, and the S1T+4 form exhibiting only splicing of exon 11). The sequences SEQ ID Nos. 28 and 30 are the corresponding amino acid sequences, respectively.

The sequences SEQ ID Nos. 31 and 33 represent the genes identified in proximity to the cellular sequence (SEQ ID No. 4) flanking one of the two sites of integration of HBV into the tumor referred to as 83T, and correspond respectively to the new gene called 83T and to the CCT7 gene. The sequences SEQ ID Nos. 32 and 34 are the polypeptide sequences corresponding to the sequences SEQ ID Nos. 31 and 33.

The sequences SEQ ID Nos. 35, 37, 39, 41 and 43 represent the genes identified in proximity to the cellular sequences flanking the sites of integration of HBV into the tumors 54T, 95T, FR2, SA1 and SA2 respectively, corresponding to the Nuclear Matrix Protein p84 gene and to the TRKB, TRUP, ST3GalVI and IP₃R₁ genes, the sequences SEQ ID Nos. 36, 38, 40, 42 and 44 being the corresponding polypeptide sequences.

The term “in proximity” is intended to mean the nucleotide sequences in which at least one of the bases is located at most at approximately 80 kb, preferably at most at approximately 150 kb, even more preferably at most approximately 200 kb, upstream or downstream of the identified cellular sequences flanking a site of integration of the HBV DNA.

A first aspect of the invention is therefore directed toward a method of detecting genes involved in oncogenesis, by identifying genes containing, or located in proximity to, a cellular nucleotide sequence flanking a site of integration of HBV into the genome. The method of detection according to the invention in particular comprises the steps consisting in-extracting DNA from a tumoral liver tissue, amplifying in vitro a nucleotide sequence at the viral DNA/cellular DNA junction, sequencing said nucleotide sequence, and identifying one or more genes comprising, or located in proximity to, said sequence thus sequenced.

The extraction of DNA from tissues is carried out according to the methods well known to those skilled in the art, for example by extraction with phenol possibly preceded by digestion with proteinase K.

The identification of the genes of interest is carried out by sequence comparison with known genes deposited in databanks or with genes predicted by sequence analysis software, such as Genescan, in genomic clones or supercontigs.

More particularly, the amplification step is based on an Alu-PCR technique using primers specific for the HBV-X gene and for the Alu repeat sequence (Minami et al., 1995). In order to avoid amplification between Alu sequences, a first amplification uses primers synthesized with dUTPs instead of dTTPs. These primers are destroyed after 10 replication cycles, in particular with the enzyme uracyl DNA glycosylase. Only the sequences specifically targeted are then amplified with a primer specific for the HBV-X region and a tag sequence introduced into the primer specific for the Alu sequence. According to a particular embodiment, the amplification involves four amplification steps using successively the pairs of primers HB1 (SEQ ID No. 47)/A5 (SEQ ID No. 48), HB2 (SEQ ID No. 49)/Tag5 (SEQ ID No. 50), 26C (SEQ ID No. 52)/Tag 5 (SEQ ID No. 50) and MX2 (SEQ ID No. 53)/Tag5 (SEQ ID No. 50).

Another aspect of the invention is directed toward an isolated nucleic acid comprising a cellular nucleotide sequence selected from SEQ ID Nos. 1, 3, 4, 5, 6, 7, 8, 9, 10, 11, 20, 23 and 24, or located in proximity to one of these sequences. In particular, this nucleic acid will correspond to a gene.

The present invention relates in particular to the use of the sequence SEQ ID No. 7 or SEQ ID No. 10, for identifying genes involved in oncogenesis. More particularly, the invention relates to the use of the sequence SEQ ID No. 7 or SEQ ID No. 10, for identifying new genes located in proximity to these sequences.

The invention relates in particular to an isolated nucleic acid comprising a nucleotide sequence selected from SEQ ID Nos. 2, 12, 14, 16, 18, 27, 29, 31, 45, 1, 3, 4, 5, 6, 7, 8, 9, 10 and 11 or a nucleotide sequence such that it encodes one of the amino acid sequences SEQ ID Nos. 13, 15, 17, 19, 28, 30 and 46.

It is understood that also included are the sequences homologous to said identified sequences, defined as:

i) sequences at least 70% similar to one of the identified sequences; or

ii) sequences which hybridize with one of said identified sequences, or the sequence complementary thereto, under stringent hybridization conditions, or

iii) sequences encoding a polypeptide, as defined below.

Preferably, a homologous nucleotide sequence according to the invention is at least 75% similar to the identified sequences, more preferably at least 85%, at least 90%.

Preferentially, such a homologous nucleotide sequence hybridizes specifically to the sequences complementary to one of the identified sequences under stringent conditions. The parameters which define the stringency conditions depend on the temperature at which 50% of the paired strands separate (Tm).

For sequences comprising more than 30 bases, Tm is defined by the equation: Tm=81.5+0.41(% G+C)+16.6Log (cation concentration)−0.63(% formamide)−(600/number of bases) (Sambrook et al., 1989).

For sequences less than 30 bases long, Tm is defined by the equation: Tm=4(G+C)+2 (A+T).

Under suitable stringency conditions, at which the aspecific sequences do not hybridize, the hybridization temperature can preferably be 5 to 10° C. below Tm, and the hybridization buffers used are preferably solutions of high ionic strength, such as a 6×SSC solution for example.

The term “similar sequences” used above refers to the perfect resemblance or identity between the nucleotides compared, and also to the imperfect resemblance which is described as similarity. This search for similarity in the nucleic acid sequences distinguishes, for example, purines and pyrimidines.

A homologous nucleotide sequence therefore includes any nucleotide sequence which differs from one of the identified sequences by mutation, insertion, deletion or substitution of one or more bases, or by the degeneracy of the genetic code.

Included among such homologous sequences are the sequences of the genes of mammals other than humans, preferably of a primate, of a bovine, a member of the sheep family or a pig, or else of a rodent, and also the allelic variants.

A subject of the invention is also isolated polypeptides encoded by these nucleic acids. In particular, the invention comprises an isolated polypeptide comprising an amino acid sequence selected from SEQ ID Nos. 13, 15, 17, 19, 28, 30, 32 and 46, or a sequence encoded by one of the nucleotide sequences SEQ ID Nos. 2, 12, 14, 16, 18, 27, 29, 31, 45, 1, 3, 4, 5, 6, 7, 8, 9, 10 and 11 or by a nucleotide sequence located in proximity to either of the sequences SEQ ID Nos. 7 and 10.

The subject of the invention is more particularly an isolated polypeptide comprising the sequence SEQ ID No. 13, identified as a new member of the family of MCM proteins.

It is understood that also included are the homologous sequences defined as:

i) the sequences at least 70% similar to one of the identified amino acid sequences; or

ii) the sequences encoded by a homologous nucleic acid sequence as defined above, i.e. a nucleic acid sequence which hybridizes with one of the identified nucleotide sequences, or the sequence complementary thereto, under stringent hybridization conditions.

Here again, the term “similar” refers to the perfect resemblance or identity between the amino acids compared, and also to the imperfect resemblance which is described as similarity. The search for similarities in a polypeptide sequence takes into account conservative substitutions, which are substitutions of amino acids of the same class, such as substitutions of amino acids with uncharged sidechains (such as asparagine, glutamine, serine, threonine or tyrosine), of amino acids with basic sidechains (such as lysine, arginine or histidine), of amino acids with acidic sidechains (such as aspartic acid or glutamic acid), or of amino acids with apolar sidechains (such as glycine, alanine, valine, leucine, isoleucine, proline, phenylalanine, methionine, tryptophan or cysteine).

More generally, the expression “homologous amino acid sequence” is therefore intended to mean any amino acid sequence which differs from the identified amino acid sequence by substitution, deletion and/or insertion of an amino acid or of a small number of amino acids, in particular by substitution of natural amino acids with unnatural amino acids or pseudo amino acids at positions such that these modifications do not significantly harm the biological activity of the polypeptide encoded.

Preferably, such a homologous amino acid sequence is at least 85% similar to the identified sequence, preferably at least 95%.

Homology is generally determined using sequence analysis software (for example, Sequence Analysis Software Package of the Genetics Computer Group, University of Wisconsin Biotechnology Center, 1710 University Avenue, Madison, Wis. 53705). Similar amino acid sequences are aligned in order to obtain the maximum degree of homology (i.e. identity or similarity, as defined above). For this purpose, it may be necessary to artificially introduce gaps into the sequence. Once the optimal alignment has been produced, the degree of homology is established by recording all the positions for which the amino acids of the two compared sequences are identical, relative to the total number of positions.

A particular aspect of the invention concerns, moreover, new forms of transcripts of the MCM8 and SERCAL genes, which differ from the transcripts encoding the MCM8 and SERCAL proteins by a differential splicing.

A subject of the invention is therefore also a nucleic acid comprising a nucleotide sequence selected from SEQ ID Nos. 14, 16, 18, 27 and 29, it being understood that all homologous sequences are included, the homology being defined in a similar way to the definition given above.

A subject of the invention is also a polypeptide comprising an amino acid sequence encoded by said nucleotide sequence, such as the sequences SEQ ID Nos. 15, 17, 19, 28 or 30.

These variant forms of MCM8 and SERCA1 are found in a set of healthy (non-tumoral) tissues and encode proteins which might modulate the activity of the wild-type MCM8 and SERCA proteins respectively.

More particularly, the expression of the truncated forms of SERCA1 may induce apoptotic cell death.

The authors of the invention have also shown that the truncated SERCA1 proteins have a dominant negative effect on both SERCA1 and SERCA2b. They have, moreover, demonstrated a regulatory role for these proteins on the calcium stores of the endoplasmic reticulum (ER), promoting calcium leakage from the ER.

The polypeptides of the present invention can be synthesized by all the methods well known to those skilled in the art. The polypeptides of the invention can, for example, be synthesized by synthetic chemistry techniques, such as Merrifield-type synthesis, which is advantageous for reasons of purity, of antigenic specificity and of undesired byproducts, and for its ease of production.

A recombinant protein can also be produced by a method in which a vector containing a nucleic acid containing one of the identified sequences or a homologous sequence is transferred into a host cell which is cultured under conditions which allow expression of the corresponding polypeptide.

The protein produced can then be recovered and purified.

The methods of purification used are known to those skilled in the art. The recombinant polypeptide obtained can be purified from cell lysates and extracts or from the culture medium supernatant, by methods used individually or in combination, such as fractionation, chromatography methods, immunoaffinity techniques using specific mono- or polyclonal antibodies, etc.

The nucleic acid sequence of interest can be inserted into an expression vector, in which it is functionally linked to elements allowing regulation of its expression, such as, in particular, promoters, activators and/or terminators of transcription.

The signals controlling the expression of the nucleotide sequences (promoters, activators, termination sequences, etc.) are selected as a function of the cellular host used. To this effect, the nucleotide sequences according to the invention can be inserted into vectors which replicate autonomy in the chosen host, or vectors which integrate in the chosen host. Such vectors will be prepared according to the methods commonly used by those skilled in the art, and the clones resulting therefrom can be introduced into a suitable host by standard methods, such as, for example, electroporation or precipitation with calcium phosphate.

The cloning and/or expression vectors as described above, containing a nucleotide sequence defined according to the invention, are also part of the present invention.

The invention is also directed toward the host cells transiently or stably transfected with these expression vectors. These cells can be obtained by introducing into prokaryotic or eukaryotic host cells a nucleotide sequence inserted into a vector as defined above, and then culturing said cells under conditions which allow replication and/or expression of the transfected nucleotide sequence.

Examples of such host cells include in particular mammalian cells, such as COS-7, 293 or MDCK cells, insect cells such as SF9 cells, bacteria such as E. coli and yeast strains such as L40 and Y90.

The nucleotide sequences of the invention may be of artificial or nonartificial origin. They may be DNA or RNA sequences obtained by screening sequence libraries using probes developed on the basis of the identified sequences. Such libraries can be prepared by conventional molecular biology techniques known to those skilled in the art.

The nucleotide sequences according to the invention can also be prepared by chemical synthesis, or else by mixed methods including chemical or enzymatic modification of sequences obtained by screening libraries.

The nucleotide sequences of the invention make it possible to produce probes or primers which hybridize specifically with one of the identified sequences according to the invention, or the strand complementary thereto. Suitable hybridization conditions correspond to the conditions of temperature and of ionic strength usually used by those skilled in the art, preferably under stringent conditions as defined above. These probes can be used as an in vitro diagnostic tool for detecting, via hybridization experiments, in particular “in situ” hybridization experiments, transcripts specific for the polypeptides of the invention in biological samples, or for demonstrating aberrant synthesis or genetic abnormalities resulting from a polymorphism, from mutations or from incorrect splicing.

The nucleic acids of the invention which are of use as probes comprise a minimum of 15 nucleotides, preferentially at least 20 nucleotides, more preferentially at least 100 nucleotides, but are preferably less than the total length of the identified cellular sequences.

The nucleic acids which are of use as primers comprise a minimum of 15 nucleotides, preferably at least 18 nucleotides, and preferentially less than 40 nucleotides.

More precisely, the subject of the invention is a nucleic acid having at least 15 nucleotides, which hybridizes specifically with one of the identified nucleic acid sequences, or the sequence complementary thereto, under stringent hybridization conditions.

Preferentially, the probes or primers of the invention are labeled, prior to their use. For this, several techniques are within the scope of those skilled in the art, such as, for example, fluorescent, radioactive, chemiluminescent or enzyme labeling. The sequences identified by the authors of the invention can, moreover, be linked to peptides so as to form PNAs (peptide nucleic acids, P E Nielsen et al., 1993) which are in particular of use as readily detectable probes.

The methods of in vitro diagnosis in which these oligonucleotides are used to detect mutation or genomic rearrangements, in the genes described here, or else to detect supernumerary copies of these genes, are included in the present invention.

Those skilled in the art are well aware of the standard methods for analyzing the DNA contained in a biological sample and for diagnosing a genetic disorder. Many strategies for genotypic analysis are available (Antonarakis et al., 1989; Cooper et al., 1991).

Preferably, use may be made of the DGGE (denaturing gradient gel electrophoresis) method, the SSCP (single-stranded conformational polymorphism) method or the DHPLC method (denaturing high performance liquid chromatography; Kuklin et al., 1997; Huber et al., 1995) for detecting an abnormality in the genes described. Such methods are preferably followed by direct sequencing. The RT-PCR method may advantageously be used to detect abnormalities in the transcripts of the genes described, since it makes it possible to visualize the consequences of a splicing mutation which causes the loss of one or more exons in transcript, or an aberrant splicing due to the activation of a cryptic site. This method is preferably also followed by direct sequencing. The methods more recently developed using DNA chips can also be used to detect an abnormality in the genes described (Bellis et al., 1997).

In general, the identified sequences, or fragments thereof, can be used to produce DNA chips of the “microarray” type (comprising cDNAs) or “DNA chips” (comprising oligonucleotides synthesized in situ or attached after synthesis).

These chips comprise a very large number of different probes (up to several tens of thousands over a surface of approximately 1 cm²) attached to an inert support, each one at a precise place. These chips can be placed together with a sample of labeled nucleic acids to be tested, and the sequences complementary to the probes pair. The sequences of the invention can thus be integrated into these chips as probes. The chips are particularly advantageous in the context of a genetic diagnosis, as seen above, but also for identifying unknown genes revealing, by hybridization with the sequences of the invention, a possible involvement in oncogenesis.

A subject of the invention is also antibodies directed against the polypeptides as defined above.

They may be poly- or monoclonal antibodies or fragments thereof, chimeric antibodies, in particular humanized antibodies or immunoconjugates.

The polyclonal antibodies can be obtained from the serum of an animal immunized against a polypeptide, according to the usual procedures.

According to one embodiment of the invention, use may be made, as antigen, of a suitable peptide fragment, as defined above, which can be coupled, via a reactive residue, to a protein or to another peptide. Rabbits are immunized with the equivalent of 1 mg of the peptide antigen according to the procedure described by Benoit et al. (1982). At four-week intervals, the animals are given injections of 200 μg of antigen and bled 10 to 14 days later. After the third injection, the antiserum is examined in order to determine its ability to bind to the antigenic peptide radiolabeled with iodine, prepared by the chloramine-T method, and is then purified by chromatography on a carboxymethyl-cellulose (CMC) ion exchange column. The antibody molecules are then collected from the mammals and isolated to the desired concentration by methods well known to those skilled in the art, for example using DEAE Sephadex to obtain the IgG fraction.

In order to increase the specificity of the polyclonal serum, the antibodies can be purified by immunoaffinity chromatography using immunizing polypeptides in solid phase. The antibody is brought into contact with the immunizing polypeptide in solid phase for a sufficient amount of time so as to immunoreact the polypeptide with the antibody molecule in order to form an immunocomplex in solid phase.

The monoclonal antibodies can be obtained according to the conventional method of hybridoma culture described by Köhler and Milstein (1975).

The antibodies or antibody fragments of the invention may, for example, be chimeric antibodies, humanized antibodies, and Fab and F(ab′)2 fragments. They may also be in the form of labeled antibodies or immunoconjugates.

The antibodies of the invention, in particular the monoclonal antibodies, can especially be used for the immunohistochemical analysis of the polypeptides on specific tissue sections, for example by immunofluorescence, gold labeling, immunoperoxidase, etc.

The antibodies thus produced can advantageously be used in any situation where the expression of the polypeptides of the invention is to be observed.

A subject of the invention is more generally the use of at least one antibody thus produced, for detecting or purifying a polypeptide as defined above in a biological sample.

A subject of the invention is also a kit for implementing this method, comprising:

at least one antibody specific to the polypeptides of the invention, optionally attached to a support;

means of revealing the formation of specific antigen/antibody complexes between the polypeptides of the invention and said antibody, and/or means of quantifying these complexes.

The invention is also directed toward a method of detecting, in vitro, polypeptides of the invention or antibodies directed against these polypeptides in a biological sample, in which said biological sample is brought into contact with respectively an antibody or a polypeptide of the invention (namely in particular a polypeptide comprising a sequence selected from SEQ ID Nos. 13, 15, 17, 19, 22, 26, 28, 30, 32, 34, 36, 38, 40, 42, 44, and 46 or encoded by a sequence comprising a nucleotide sequence selected from SEQ ID Nos. 43, 1, 3, 4, 5, 6, 7, 8, 9, 10, 11, 20, 23, 24, 2, 12, 14, 16, 18, 21, 25, 27, 29, 31, 33, 35, 37, 41 and 45 or located in proximity to SEQ ID Nos. 1, 3, 4, 5, 6, 7, 8, 9, 10, 11, 20, 23 and 24) or an epitope fragment thereof, and the formation of immunocomplexes, revealing the presence of polypeptides of the invention or, against these polypeptides, of antibodies, respectively, in the biological sample is observed.

Another aspect of the invention is directed toward the diagnostic and therapeutic uses relating to the sequences identified by the inventors in proximity to the HBV integration sites, and also corresponding polypeptides and antibodies.

The authors of the present invention have in fact shown that all of these genes are involved in oncogenesis, and more particularly in hepatic carcinogenesis.

These results open up multiple perspectives in the field of cancerology.

The nucleic acids comprising at least one of the sequences of the genes described above, or those homologous thereto, are of use for detecting an abnormality in these genes or in their transcripts.

A subject of the invention is, consequently, a method of in vitro diagnosis of a tumor or of a predisposition to developing a tumor, comprising the steps consisting in:

bringing a biological sample containing DNA or RNA into contact with specific oligonucleotides allowing the amplification of all or part of a gene as described above (namely in particular a gene comprising a sequence selected from SEQ ID Nos. 43, 1, 3, 7, 9, 10, 11, 20, 24, 2, 12, 14, 16, 18, 21, 25, 27, 29, 31, 33, 35, 37, 39, 41 and 45) or of its transcript;

amplifying said DNA or RNA;

detecting the amplification products;

comparing the amplification products obtained to those obtained with a control sample, and detecting in this way a possible abnormality in said gene or in its transcript or else an abnormal number of copies of said gene, indicating a tumor or a predisposition to developing a tumor.

The biological sample in question may in particular be blood, or a tissue fragment taken from a patient.

Since the expression of these genes is found on cancer cells, the methods for evaluating the expression of the corresponding polypeptides, using samples of suspect tissues from patients, are also particularly advantageous in diagnostic tests, in anatomopathology.

These methods may be directed towards detecting the mRNA encoding these polypeptides, or these polypeptides themselves.

According to the first embodiment, the invention relates to a method of in vitro diagnosis of a tumor or of a predisposition to developing a tumor, comprising the steps consisting in

bringing a biological sample containing mRNA obtained from a sample of suspect cells from a patient into contact with specific oligonucleotides allowing the amplification of all or part of the transcript of the targeted genes (namely in particular a gene comprising a sequence selected from SEQ ID Nos. 43, 1, 3, 7, 9, 10, 11, 20, 24, 2, 12, 14, 16, 18, 21, 25, 27, 29, 31, 33, 35, 37, 39, 41 and 45);

amplifying said transcript;

detecting and quantifying the amplification products;

a modification of the level of transcript of the targeted genes compared to the normal control being an indicator of a tumor or of a predisposition to developing a tumor.

According to a second embodiment, the invention relates to an in vitro method for diagnosing a tumor or a predisposition to developing a tumor, comprising detecting or measuring the level of expression of the polypeptides described above in a biological sample obtained, for example, from a sample of suspect cells from a patient.

The presence of the polypeptides or of the genes transcribed from the genes in amounts different from the normal amount can be correlated with a more or less serious prognosis, in terms of aggressiveness of the tumor, for example.

In certain circumstances, in which modified expression of the polypeptides described above, correlated with a tumor phenotype, is, for example, observed, restoration or stimulation of the activity of said wild-type polypeptides may be sought.

The polypeptides described above or the corresponding nucleic acids may then be of use as a medicinal product.

A subject of the invention is therefore also a pharmaceutical composition comprising a polypeptide as defined above or a nucleic acid encoding said polypeptide, in combination with a pharmaceutically acceptable vehicle.

The methods of administration, the dosages and the pharmaceutical forms of the pharmaceutical compositions according to the invention, containing at least one polypeptide, can be determined in the usual way by those skilled in the art, and in particular according to the criteria generally taken into account in establishing a suitable therapeutic treatment for a patient, such as, for example, the age or the bodyweight of the patient, the seriousness of his or her general condition, the tolerance to the treatment and the side effects noted, etc.

In general, a therapeutically or prophylactically effective amount ranging from approximately 0.1 μg to approximately 1 mg can be administered to human adults.

A subject of the invention is also a pharmaceutical composition comprising a nucleic acid as defined above, and a pharmaceutically acceptable vehicle, said composition being intended to be used in gene therapy. The nucleic acid preferably inserted into a generally viral vector (such as adenoviruses and retroviruses) can be administered in naked form, free of any vehicle promoting transfer to the target cell, such as anionic liposomes, cationic lipids, microparticles, for example gold microparticles, precipitating agents, for example calcium phosphate, or any other agent promoting transfection. In this case, the polynucleotide can be simply diluted in a physiologically acceptable solution, such as a sterile solution or a sterile buffer solution, in the presence or absence of a vehicle.

Alternatively, a nucleic acid according to the invention can be combined with agents which facilitate the transfection. It can be, inter alia, (i) combined with a chemical agent which modifies the cellular permeability, such as bupivacaine; (ii) encapsulated in liposomes, optionally in the presence of additional substances which facilitate transfection; or (iii) combined with cationic lipids or microparticles of silica, of gold or of tungsten. When the nucleic acid constructs of the invention coat microparticles, they can be injected intradermally or intraepidermally using the “gene gun” technique (WO 94/24263).

The amount to be used as medicinal product depends in particular on the nucleic acid construct itself, on the individual to which this nucleic acid is administered, on the method of administration and on the type of formulation, and on the pathological condition. In general, a therapeutically or prophylactically effective amount ranging from approximately 0.1 μg to approximately 1 mg, preferably from approximately 1 μg to approximately 800 μg, and preferentially from approximately 25 μg to approximately 250 μg, can be administered to human adults.

The nucleic acid constructs of the invention can be administered by any conventional route of administration, such as in particular parenterally. The choice of the route of administration depends in particular on the formulation chosen. An administration targeted to the site of the targeted tumors may be particularly advantageous.

Finally, the subject of the invention is therefore a method of therapeutic treatment, in which an effective amount of a polypeptide as defined above or a nucleic acid encoding this polypeptide, in the case of a gene therapy, is administered to a patient requiring such a treatment.

The patient targeted is generally a human, but the application may also be extended to any mammal as appropriate.

Conversely, in other circumstances where overexpression of the polypeptides described above, in correlation with a tumor phenotype, is observed, for example, blocking or inhibiting the activity of said polypeptides may be sought.

A subject of the invention is therefore also a pharmaceutical composition comprising an antibody directed against said polypeptide, in combination with a pharmaceutically acceptable vehicle.

A subject of the invention is also a pharmaceutical composition comprising a nucleic acid comprising an antisense sequence which blocks the expression of the genes described above, in combination with a pharmaceutically acceptable vehicle.

The formulation of these pharmaceutical compositions and the associated dosage are within the scope of those skilled in the art, in view in particular of the information given above, concerning the pharmaceutical compositions based on polypeptides or a nucleic acid encoding the polypeptides.

More precisely, the subject of the invention is the use of a nucleic acid comprising a sequence selected from SEQ ID Nos. 43, 1, 3, 7, 9, 10, 11, 20, 24, 2, 12, 14, 16, 18, 21, 25, 27, 29, 31, 33, 35, 37, 39, 41 and 45, of an antisense of said nucleic acid, of a polypeptide encoded by said nucleic acid, or of an antibody against said polypeptide, for producing a medicinal product intended to treat tumors.

In general, the sequences identified by the invention as being involved in oncogenesis, or the corresponding polypeptides or antibodies, can be advantageously used as tools to search for (screening and identifying) therapeutic agents exhibiting simulatory or inhibitory activity targeted toward them.

The following examples and figures illustrate the invention without limiting the scope thereof:

### FIGURE LEGENDS

FIG. 1 represents the structure of the integration of the HBV DNA in 5 genes, the codes 86T, 100T, 77T, 83T and FR7 being the codes of the HCC tumors of various patients, present in table 1. The open clear box represents the HBV sequence, HBVX, the region X of the HGB genome. The number above the box indicates the last base of HBV before the cellular/HBV junction. The numbering of the HBV genome begins from the hypothetical EcoR1 site of subtype adw. The hatched boxes represent the regions homologous with respect to the sequences of the databases, the darkened boxes representing the coding sequences. The arrows in bold indicate the direction of the ORF. The double-headed arrow marks the length of the cellular sequence obtained by Alu-PCR. The dashed line indicates the distance of 10 800 base pairs between the sequence identified by the inventors and the end of the coding sequence for hTERT.

FIG. 2A represents a structure of the variant cDNAs of SERCA1 with a splicing of exon 11 and an alternative splicing of exon 4, according to cloning in a normal liver. The splicing of exon 11 produces a mutated sequence of 22 amino acids (black box), a premature stop codon appearing in exon 12.

The spliced transcripts of SERCA1 therefore encode proteins truncated in their C-terminal portion.

FIG. 2B represents a predicted structure of the SERCA1 and S1T proteins. The numbers under the drawings indicate the transmembrane domains.

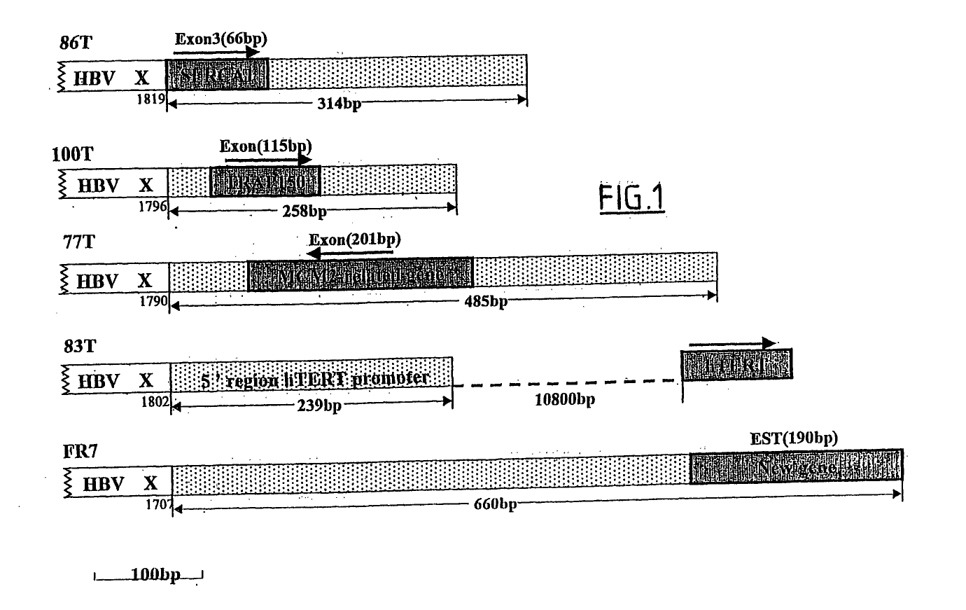

| | |
|---|---|
| D351: | phosphorylatable aparagine 351. |
| (●): | calcium-binding transmembrane residues. |
| (◯): | calcium-binding cytoplasmic residues. |

The mutated C-terminal sequence is represented as a dashed line (22 amino acids) and the peptide (35 amino acids) encoded for exon 4 is in gray.

### EXAMPLES

### Example 1

### Identification of the Viral DNA/Cellular DNA Junctions by the Alu-PCR Technique

The authors of the present invention developed a new technical approach based on a PCR method using primers specific for Alu sequences and primers specific for the HBV genome (Minami et al., 1995).

This method envisions amplifying the DNA extracted from liver tumors, with the primer HB1 (5′ACAUGAACCUUUACCCCGUUGC3′, SEQ ID No. 47) and the primer A5 (5′CAGUGCCAAGUGUUUGCUGACGCCAAAGUGCUGGGAUUACAG3′, SEQ ID No. 48). The amplification is carried out in a final volume of 50 μl, in 25 mM Tris buffer, pH 8.9, 40 mM potassium acetate, 2.5 mM magnesium chloride and 4% glycerol, with 10 pmol and 100 pmol of primers A5 and HB1 respectively. Each of the dNTPs is present at the concentration of 200 μm. The amplification is carried out using the enzymes Taq polymerase (Gibco BRL, MD, USA), 1 unit, and Vent exo+polymerase (New England Biolabs, MA, USA), 0.04 units. The PCR conditions are as follows: denaturation 30 s at 94° C., hybridization 30 s at 59° C., elongation 3 min at 70° C.

After 10 amplification cycles, the primers HB1 and A5—synthesized with dUTPs in place of dTTPs—are destroyed by adding 1 unit of the enzyme uracyl DNA glycosylase and incubating at 37° C. for 30 min.

After 10 min at 94° C., the amplification is then continued with 10 pmol of each of the primers HB2 (5′GCGCTGCAGTGCCAAGTGTTTGCTGACGC3′, SEQ ID No. 49) and Tag5 (5′CAAGTGTTTGCTGACGCCAAAG3′, SEQ ID No. 50) according to the following conditions:

| | |
|---|---|
| 20 cycle | 30 s at 94° C., 30 s at 65° C., 3 min at |
| | 70° C., with decrease in the temperature of |
| | hybridization of the primers of 1° C. every |
| | two cycles, to 55° C. |
| 19 cycles | 30 s at 94° C., 30 s at 55° C., 3 min at 70° C. |
| final cycle | 30 s at 94° C., 30 s at 55° C., 8 min at 72° C. |

1 μl of this amplification is subjected to a PCR using the internal primer MD60 (5′CTGCCGATCCATACTGCGGAAC3′, SEQ ID No. 51) and the primer Tag5.

This method, described in the article Minami et al. (1995), did not make it possible to isolate a large number of viral DNA/cellular DNA junctions. The inventors then modified the technique. Once the HB2-Tag5 amplification had been carried out, as described in Minami et al. 1995, 2 μl of this amplification was used to carry out another amplification with the primer 26C (K. Poussin et al., 1999; SEQ ID No. 52) and the primer Tag5. The 26C-Tag5 amplification is carried out exactly like the HB2-Tag5 amplification. 2 μl of the 26C-Tag5 amplification were then used to carry out an amplification using the primers MX2 (5′TGCCCAAGGTCTTACATAAGAGGA3′, SEQ ID No. 53) and Tag5.

The sequence MX2, highly conserved in the various subtypes of HBV, is located in the HBV genome at position 1639 (from nucleotide 1639 to nucleotide 1662). This primer allowed the inventors to isolate a large number of viral DNA/cellular DNA junctions by virtue of its effectiveness and its position. Specifically, the HBV-Alu technique isolates quite large fragments (1 Kb or more). In order for it to be effective, it is therefore necessary to use a primer on the HBV genome which is as close as possible to the junction but not beyond. Now, this task is difficult since the integration points of the viral genome are not known. The identification of this primer is therefore derived from the vast experience of the inventors in this field and from the analysis of several integration sites.

The MX2-Tag5 amplification is carried out in a final reaction volume of 100 μl, in a 25 mM Tris buffer, pH 8.9, 40 mM potassium acetate, 2.5 mM magnesium chloride and 4% glycerol. The molarity of each primer is 10 pmol and each of the dNTPs is present at the concentration of 250 μM. The amplification is carried out using the enzymes Taq polymerase (Gibco BRL, MD, USA), 1.2 units, plus Vent exo+polymerase (New England Biolabs, MA, USA).

The amplification is carried out in a Perkin-Elmer thermal Cycler 9600 (Emeryville, Calif., USA) in the following way:

| | |
|---|---|
| First cycle | 2 min at 94° C., 30 s at 63° C., 4 min at 72° C. |
| 14 cycles | 30 s at 94° C., 30 s at 67° C., 4 min at 72° C., |
| | with decrease in the temperature of |
| | hybridization of the primers of 1° C. at |
| | each cycle, to 54° C. |
| 24 cycles | 30 s at 94° C., 30 s at 54° C., 4 min at 72° C. |
| Final cycle | 30 s at 94° C., 30 s at 54° C., 10 min at 72° C. |

### Example 2

### Identification of Genes Involved in Oncogenesis by Insertion Mutation by the Hepatitis B Virus

The DNA was extracted from tumoral and nontumoral tissues as described in Paterlini et al. 1990. In order to amplify the cellular DNA/HBV junctions in the tumoral tissue, an Alu-PCR was carried out as described above.

Table 1 given below shows the result of the screening of 45 patients suffering from hepatic carcinomas positive for the hepatitis C virus, among which 21 cellular DNA/HBV integration sites were isolated by the inventors.

TABLE 1
HBV serology and liver histology of the patients analyzed

| | Histology in the | | | Cellular flanking |
|---|---|---|---|---|
| HCC code | nontumoral tissues | HbsAg | Anti-HBs | sequences (bp) |
| 54T | MLC | + | − | 475 |
| 63T | MLC | + | − | 606 |
| 77T | CH | + | − | 485 |
| 83T | LC | + | − | 673 |
| | | | | 239 |
| 86T | CH | − | + | 314 |
| 95T | LC | + | − | 355 |
| 100T | MLC | + | − | 258 |
| 100T | CH | + | − | 502 |
| FR2 | LC | + | − | 1220 |
| FR3 | LC | + | − | 1349 |
| FR7 | LC | + | − | 660 |
| SA1 | CH | + | − | 366 |
| SA2 | LC | + | − | 511 |
| | | | | 591 |
| SA5 | LC | + | + | 586 |
| GR1 | LC | − | + | 282 |
| GR2 | LC | + | − | 271 |
| | | | | 780 |
| GR3 | LC | + | − | 886 |
| GR10 | | + | − | 1880 |

MLC: minimal liver changes,
CH: active chronic hepatitis,
LC: liver cirrhosis,
HbsAg: hepatitis B surface antigen,
anti-HBs: antibody against the hepatitis B surface antigen

In one tumor (86T), the integration of the HBV DNA occurred in phase in the third exon of the SERCA1 gene (accession number U96773, SEQ ID No. 24). The SERCA proteins play a key role in the regulation of cellular calcium (Pozzan et al., 1994), which in turn acts as an intracellular messenger involved in a broad panel of basic or specialized cellular activities, including cell proliferation and cell death (Berridge, 1998). In the tumor, the integration of HBV produces cis-activation of HBV-X/SERCA1 fusion transcripts (Chami et al., 2000). In vitro expression of the HBV/SERCA1 transcripts induces calcium depletion in the endoplasmic reticulum and apoptosis. The inventors' results show, for the first time, a mutation of the SERCA gene in a malignant tumor pathology.

In a second tumor (100T), the integration of the HBV DNA was identified in proximity to a cellular sequence of 258 base pairs (SEQ ID No. 20) containing a sequence of 115 base pairs identical to the TRAP 150 gene. SEQ ID No. 20 corresponds to nucleotides 103283 to 103541 of the genomic sequence of TRAP150 (accession number, AL360074). SEQ ID No. 21 corresponds to the cDNA of TRAP150 (accession number AF117756, nucleotides 2119 to 2233). The HBV genome and the TRAP150 ORF are in the same orientation. The TRAP proteins contribute to the protein complex which coactivates the nuclear thyroid hormone receptor in the presence of the ligand (Ito, 1999). The thyroid hormone is one of the major regulatory agents of hepatic cell proliferation (Lin et al., 1999). However, the precise biological role of the gene of the human TRAP150 protein is still unknown.

In another tumor, 77T, the authors of the invention found that the integration of the HBV DNA occurred in a new gene of the MCM (for “minichromosome maintenance”, Bik K. Tye, 1999) gene family, called MCM8 gene. The HBV DNA was in fact located in proximity to a sequence of 485 base pairs (SEQ ID No. 11) identical to a portion of the gene-like MCM 2/3/5 sequence on chromosome 20p12.3-13. SEQ ID No. 11 corresponds to nucleotides 51574 to 52059 of the gene-like MCM 2/3/5 sequence (accession number: AL035461). Software analysis of the sequence of the clone with gene prediction programs and the experimental data of the inventors indicate that this sequence encodes a new member of the MCM protein family, called MCM8. The HBV genome exhibits an orientation opposite to that of the MCM8 ORF. The MCM proteins are a family of proteins including six members (MCM2 to 7) which are highly conserved from yeast to humans. The six MCM proteins form a complex which has DNA helicase activity and are involved in the control of DNA replication once per cycle (Kearsey et al., 1998). The MCM proteins are highly expressed in proliferative and neoplastic cells. The cDNA of this new human MCM8 gene was cloned by the inventors from normal liver (SEQ ID No. 12). A monoclonal antibody directed against an N-terminal peptide sequence specific for MCM8 was produced so as to study the expression of this new gene in the tumor and the adjacent hepatic tissue. Western blotting analysis showed that a truncated form of MCM8 (33kD) is specifically expressed in the tumoral tissue (77T) compared to the healthy tissue. The size of this protein is compatible with a form of the MCM8 protein truncated in its C-terminal portion due to a premature stop codon associated with the integration of the HBV DNA (predicted size: 32.8 kDa).

In another carcinoma (83T), the HBV DNA integrated in proximity to a cellular sequence of 239 base pairs (SEQ ID No. 23) identical to a sequence located upstream of the promoter of the hTERT gene (SEQ ID No. 23, bases 240 to 478 of the genomic sequence of hTERT (accession number: AF128893)). The HBV genome and the hTERT gene ORF have opposite orientations. Telomerases are expressed in the majority of human malignant tumors and are absent from differentiated somatic tissues (Urquidi et al., 2000). Normal primary cells can be immortalized by stable transfection with the telomerase gene. The activation of telomerases is one of the modifications required for the malignant transformation of human fibroblasts. Western blotting analysis of the tumor 83T using an antibody directed against the hTERT protein shows overexpression of hTERT in the tumoral tissues compared to the nontumoral tissues. It also shows overexpression of an hTERT protein which is larger in size (approximately 170 kD) compared to the wild-type hTERT protein. This observation therefore confirms the inventors' hypothesis according to which the integration of the HBV genome occurs in genes involved in tumorigenic processes.

In this same carcinoma 83T, a second HBV integration site was identified on chromosome 2p24.-24.3. The isolated cellular sequence is positioned between nucleotides 883565 and 884238 in the supercontig NT025651 (SEQ ID No. 4). This sequence is located 2 kb upstream of the ATG codon of a new predictive gene containing a homeobox-type domain. This new gene, called 83T gene (SEQ ID No. 31), is located between bases 881787 (ATG) and 812959 (polyA sequence) of this supercontig NT025651. The HBV genome and the 83T gene ORF exhibit an opposite orientation. Analysis using the Genescan software predicts that the HBV integration site is located between the ATG initiation codon and the 83T gene promoter (predicted position of the promoter: bases 893015 to 893054). The corresponding 83T protein is said to contain a DNA-binding domain which exhibits 70% homology (56% identity) with the human homeobox protein EMX2, and 70% homology (54% identity) with the EMXl protein. Homeobox genes are genes which are highly conserved in evolutionary terms and which encode transcription factors involved in development.

This second integration site was located 53 kb upstream of the gene encoding the CCT7 chaperone protein (Chaperonin containing TCP1, subunit7). This gene is positioned between nucleotides 771916 and 790598 of the supercontig NT025651 (SEQ ID No. 33). The ORFs of the HBV genome and of CCT7 are of the same orientation. The CCT7 protein is involved in a TCP1 (tailless complex polypeptide 1) complex, which is itself part of a heterooligomeric complex TRiC (TCP1-ring complex) which plays a part in the folding of many cellular proteins (McCallum et al., 2000). An essential role of the TRiC complex has been demonstrated for cyclin E maturation in human cells in culture (Wo et al., 1998).

In the tumor 54T, the HBV DNA integrated into chromosome 18p11.3, in an intronic sequence (bases 222267 to 222742 of the supercontig NT011005, SEQ ID No. 3) of the Nuclear Matrix Protein p84. The orientation of the HBV genome and of the ORF of the NMP p84 gene is the same. The Nuclear Matrix Protein p84 gene (accession number: XM 008756, SEQ ID No. 35) encodes a nuclear protein which binds the p110^{RB} protein in its N-terminal region (Durfee et al., 1994). This binding is thought to participate in concentrating the p110^{RB} protein in certain subnuclear regions.

In the tumor 95T, HBV integrates into chromosome 9q21.1, 13 kb downstream of the sequence encoding Neurotopic Tyrosine Receptor Kinase 2 (NTRK2 or TRKB, SEQ ID No. 37). The isolated cellular sequence is positioned between nucleotide 3194297 and 394652 of the supercontig NT023935 (SEQ ID No. 5). The orientation of the HBV genome and of the cellular gene ORF is the same. The trk family of neurotrophin receptors (trkA, trkB, trkC) promotes the survival, growth and differentiation of the neuronal and non-neuronal tissues. The TrkB protein is expressed in neuroendocrine-type cells in the small intestine and colon, in the alpha cells of the pancreas, in the monocytes and macrophages of the lymph nodes and of the spleen, and in the granular layers of the epidermis (Shibayama and Koizumi, 1996). Expression of the TrkB protein has been associated with an unfavorable progression of Wilms tumors and of neuroblastomas. TkrB is, moreover, expressed in cancerous prostate cells but not in normal cells. The signaling pathway downstream of the trk receptors involves the cascade of MAPK activation through the Shc, activated Ras, ERK-1 and ERK-2 genes, and the PLC-gammal transduction pathway (Sugimoto et al., 2001).

The integration of HBV in the tumor FR2 occurs in chromosome 14q24.2. The isolated cellular sequence is delimited by positions 559079 and 600299 of the supercontig NT010159 (SEQ ID No. 6). It is located 8 kb downstream of the gene encoding the TRUP protein (thyroid hormone uncoupling protein; Burris et al., 1995; accession number: M36072, SEQ ID No. 39), also called Ribosomal Protein L7a. The orientation of the HBV genome and the TRUP gene ORF is the same. The TRUP gene is supposed to be a pseudogene insofar as its sequence, which lacks an intron, comprises only one exon. A homologous gene with intronic sequences is located on chromosome 9q33-q34 and encodes a protein of 266 amino acids. The TRUP protein interacts with the hinge region and the N-terminal portion of the thyroid hormone receptor binding domain. It acts on the thyroid hormone receptor and the retinoic acid receptor by blocking their binding to DNA. TRUP therefore represents a regulatory protein which modulates the transcriptional activity of the nuclear hormone receptor superfamily (Burris et al., 1995). Differential display analyses have shown that the gene of ribosomal protein L7a is expressed in malignant brain tumors (Kroes et al., 2000). A subtractive hybridization analysis relating to 36 cases of human colorectal cancer has, moreover, indicated that this same gene is overexpressed in 72% of cases (Wang et al., 2000). Ribosomal protein L7a is addressed, via its domain II, to the nucleolus (Russo et al., 1997). Finally, the protooncogene trk2H becomes activated, in a cell line derived from a breast tumor, through its fusion with the large subunit of ribosomal protein L7a (Ziemiecki et al., 1990).

In the tumor SA1, the HBV DNA integrates into chromosome 3q11.2, 3 kb upstream of the alpha 2,3-sialyltransferase gene (ST3GalVI, SEQ ID No. 41). The isolated cellular sequence is positioned between nucleotides 29061 and 28695 of the supercontig NT005494 (SEQ ID No. 8). The HBV genome exhibits an orientation opposite to the ORF of the ST3GalVI gene. Alpha 2,3-sialyltransferases are involved in protein glycosylation. The genes of this family are hyperexpressed in several types of tumor: breast, stomach and colon cancers, and liver metastases (Petretti et al., 2000). In human colorectal carcinomas, expression of the gene is correlated with the malignant progression of the disease (Schneider et al., 2001). Suppression of sialyltransferase expression with an antisense DNA strategy causes a decrease in the invasiveness of human colon cancer cells in vitro (Zhu et al., 2001).

Integration of the HBV genome into the tumor SA2 occurs in chromosome 3p25, in the 38^{th} intron of the IPR₃R₁ gene. The isolated cellular sequence is delimited by nucleotides 4987889 to 5009109 of the supercontig NT005927 (SEQ ID No. 9). The ORF of the inositol 1,4,5-triphosphate receptor type 1 gene (IP₃R₁, SEQ ID No. 43) and the HBV genome have the same orientation.

In another tumor (FR7), the HBV DNA was integrated in proximity to a cellular sequence of 660 base pairs (SEQ ID No. 1) identical to a fragment of the genomic clone AC009318, on chromosome 12p (bases 112312-112971). A 190 base pair fragment of this sequence (SEQ ID No. 2) is identical to human ESTS derived from fetal tissues (accession numbers N67205 and H16791) and tumoral tissues (neuroblastoma and breast cancer; Cancer Genome Anatomy Project, accession numbers AI361463 and AA996057). Analysis using the Genescan software showed that the predicted “FR7” gene exhibits no homology with a known gene or domain. This analysis predicts an mRNA 3847 base pairs long. The FR7 gene contains successively the ESTs AK001927 (positions 2128117 to 2128688), BG741742 (positions 2128634 to 2129361) and BF572281 (3′ end at position 2130561), said ESTs being located by their position on the supercontig NT009622. SEQ ID No. 45, delimited by positions 2128117 to 2130561 of the supercontig NT009622, therefore corresponds to a partial sequence of the FR7 gene. According to this study, HBV integrates into the sequence corresponding to the EST BG741742 (position 2129217 on the supercontig NT009622). The ORFs of the HBV genome and of FR7 have the same orientation. Northern blotting experiments showed expression of this new gene in a normal liver of a human adult. Another Northern blotting experiment revealed the presence of bands of abnormal size in the FR7 tumoral tissue, compared to those identified in normal liver. This study showed the expression of a chimeric transcript X/FR7 expressed in the tumor FR7 (Gozuacik et al., Oncogene, in press).

The authors of the invention thus found that integration of the hepatitis B virus into cellular hepatic carcinoma genes occurred both in cirrhotic and noncirrhotic livers and both in patients positive and negetive for the hepatitis B surface antigen, indicating the general interest of this viral integration in hepatic cell transformation.

### Example 3

### Demonstration of Various SERCA1 Transcripts

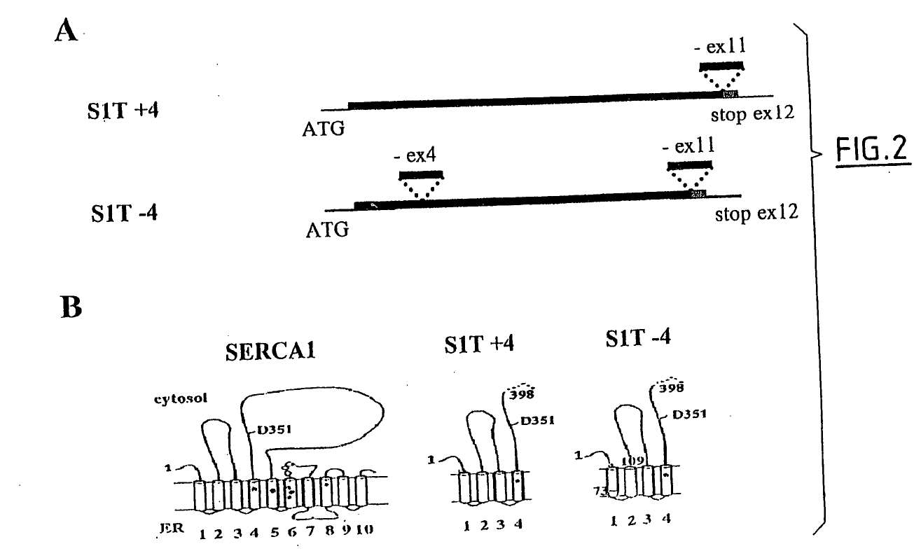

The authors of the invention cloned the SERCA1 transcripts from normal livers and obtained 25 clones. Eight of them were characterized by splicing of exon 11, including two with splicing of both exon 11 and exon 4. The splicing of exon 11 produces a shift of 22 amino acids followed by a stop codon in exon 12 (FIG. 2A). These spliced transcripts encode proteins truncated in the C-terminal portion (SERCA1-T), in which six putative transmembrane segments of SERCA1 (M5-10) are missing, including 4 of the 5 calcium-binding residues (Glu-771, Asn-796, Thr-799 and Asp-800) and also the cytoplasmic loop between transmembrane segments 6 and 7 which also controls calcium binding. Thus, these truncated proteins cannot function as calcium pumps (FIG. 2B). In addition the S1T-4 form does not contain the peptide (amino acids 74-108) encoding the second putative transmembrane segment (M2), nor the last five C-terminal residues of M1. The expected size of the S1T+4 and S1T-4 proteins is 46 and 43 kDa, respectively.

A peptide corresponding to the 10 C-terminal amino acids of the SERCA1-T truncated proteins (RQHSPPWWRR) was synthesized (Sigma-Genosis Ltd, GB). After immunizations in rabbits, and then collection of the sera, a polyclonal antibody, called anti-SERCA1-T, specific for the SERCA1-T truncated proteins, was purified by affinity chromatography against the synthetic peptide. This anti-SERCA1-T antibody does not recognize the untruncated SERCA1 proteins.

An RT-PCR analysis of the spliced transcripts of SERCA1 was carried out on various human adult and fetal tissues. This analysis revealed that the SERCA1-T transcripts are expressed in various adult human tissues (pancreas, liver, kidney, lung and placenta, and also in the spleen and the thymus) and in various fetal human tissues (kidney, liver, brain and thymus). They are not expressed in adult skeletal muscle, heart and brain, or in fetal skeletal muscle and fetal heart. The SERCA1-T/SERCA1 ratio is significantly increased in fetal liver and kidney in comparison with the corresponding adult tissues.

An RT-PCR analysis of the spliced transcripts of SERCA1 was also carried out on various tumoral tissues of human hepatocarcinomas and on the adjacent nontumoral tissues, and also on two different normal livers and on three cell lines derived from hepatic cells. In 7 of 11 pairs of tumoral tissues of hepatocarcinomas and adjacent nontumoral tissues, the authors of the invention observed a significantly increased SERCA1-T/SERCA1 ratio in the tumoral tissues versus the nontumoral tissues. The same result was obtained in the Hep3B, HepG2 and Huh7 cell lines, in comparison with the normal liver tissues.

The use of the anti-SERCA1-T polyclonal antibody made it possible to confirm the protein expression of the truncated forms of SERCA1 in the human tumor lines CCL13 and T47D, just as in untransfected primary human Hs27 cells.

The truncated SERCA1 protein could be detected in the microsomal fraction of transiently transfected cells, by western blotting. The SERCA1-T protein was detected as a monomer (46kDa) under denaturing conditions (sample heated and treated with urea), whereas SERCA1-T dimers (92kDa) were demonstrated under less denaturing conditions (sample heated but without urea). Immunohistochemical analyses of the transfected cells were carried out with the anti-SERCA1-T antibody and made it possible to detect a reticular location in the cells transfected with S1T+4.

The authors of the invention then analyzed the subcellular location of the SERCA1-T proteins. These analyses are based on the study of the colocalization of SERCA1-T with the endogenous SERCA2b protein which is an effective marker of localization in the endoplasmic reticulum. The SERCA1-T and SERCA1 constructs were cloned into expression vectors, as a fusion with the GFP sequence. SERCA1 fused with GFP was used as a control. The subcellular distribution of the proteins encoded was studied in transiently transfected HuH7 cells, using immunofluorescence and scanning confocal microscopy. The authors of the invention were thus able to show the colocalization of S1T+4 and S1T-4 fused to GFP with endogenous SERCA2b stained with a monoclonal anti-SERCA2 antibody (clone IID8).

Overexpression of the SERCA-T proteins induces apoptosis in three different hepatic cell-derived cell lines (CCL13, HuH7 and HepG2). The morphological analysis was based on counting the apoptotic bodies in the cells expressing GFP. In the transiently transfected HuH7 cells, the percentage of apoptotic bodies with S1T+4 and S1T-4 is significantly greater than in the controls (cells transfected with GFP and SERCA1). Similar results were obtained in CCL13 cells. This result was confirmed by flow cytometry experiments based on analysis of the mitochondrial structures using staining with nonylacridine orange (NAO). In comparison to the controls (NTC or pcDNA3 transfected cells), the cells transfected with S1T+4 and S1T-4 showed a significantly higher number of apoptotic cells characterized by a smaller cell size associated with a lower incorporation of NAO.

To investigate the effects of the SERCA-T proteins on calcium homeostasis in the endoplasmic reticulum (ER) lumen, the authors of the invention selectively measured the calcium concentration in the ER using targeted ER-AEQ chimeras (aequorin). This analysis was carried out in three different cell lines (HUH7, CCL13 and Hela cells) and was based on cotransfection of the ER-AEQ construct and of the constructs encoding S1T+4 or S1T-4. To obtain a quantitative estimation of the Ca2+ concentration in the endoplasmic reticulum lumen, the Ca2+ concentration was decreased during both the reconstitution of the AEQ with coelenterazine and the initial phase of perfusion. Under these conditions, the Ca2+ concentration was 10 μM in the endoplasmic reticulum. When the calcium concentration of the perfusion medium was modified to 1 mM, the calcium concentration of the endoplasmic reticulum lumen gradually increased to reach a plateau value. The results of this experiment are given in table 2 below.

TABLE 2
Calcium content in the ER

| Construct | [calcium] ER (μM) |
|---|---|
| Control HuH7 | 330.24 +/− 71.62 (n = 14) |
| S1T + 4 HuH7 | 173.52 +/− 58.16 (n = 10) |
| S1T − 4 Huh7 | 241.14 +/− 77.68 (n = 8) |
| Control CCL13 | 284.15 +/− 31.46 (n = 4) |
| S1T + 4 CCL13 | 189.73 +/− 34.52 (n = 6) |
| S1T − 4 CCL13 | 244.8 +/− 24.39 (n = 5) |
| Control Hela | 339.85 +/− 47.56 (n = 7) |
| S1T + 4 Hela | 268.92 +/− 32.55 (n = 14) |
| S1T − 4 Hela | 287.11 +/− 44.08 (n = 9) |

In comparison with the control cells, the calcium content of the endoplasmic reticulum was therefore significantly decreased in the cells transfected with S1T+4 and S1T-4 compared to the control cells. In these cells expressing S1T+4 and S1T-4, the Ca2+ ion efflux is significantly more rapid than in the control cells.

The truncated SERCA1s determine a decrease in the calcium in the calcium stores of the ER in vitro.

The authors of the invention postulated that the truncated SERCA1 proteins might have a dominant negative effect on the endogenous SERCA proteins and modulate their pump function.

This dominant negative effect of the truncated SERCA1 proteins was studied in HUH7 and Hela cells in cotransfection tests with SERCA1 or SERCA2. This study was based on measuring the calcium content of the ER using the ER-AEQ construct.

TABLE 3
Table 3 gives the results obtained in the HuH7 cells:

| | | % ER [CA²⁺] |
|---|---|---|
| | | accumulation compared |
| Construct | [calcium] ER (μM) | to the control |
| Control | 330 +/− 72 (n = 14) | 100 |
| S1T + 4 | 174 +/− 58 (n = 10) | 53 |
| S1T − 4 | 241 +/− 78 (n = 8) | 73 |
| SERCA1 | 448 +/− 81 (n = 13) | 136 |
| SERCA1 + S1T + 4 | 297 +/− 78 (n = 11) | 90 |
| SERCA1 + S1T − 4 | 343 +/− 99 (n = 10) | 103 |
| SERCA2 | 687 +/− 105 (n = 6) | 208 |
| SERCA2 + S1T + 4 | 434 +/− 131 (n = 4) | 131 |
| SERCA2 + S1T − 4 | 445 +/− 61 (n = 4) | 134 |

In the HUH7s, the value of the plateau in the cells transfected with SERCA1 and SERCA2 is increased, to reach 136% and 208%, compared to the percentage accumulation in the control cells (100%). The cotransfection of SERCA1 with S1T+4 or S1T-4 lowers the percentage accumulation to 90% and to 103%, respectively, compared to the control (table 3). The cotransfection of SERCA2 with S1T+4 or S1T-4 lowers the percentage accumulation to 131% and to 134%, respectively, compared to the control. Similar results were obtained with the Hela cells.

This result clearly shows a dominant negative effect of the truncated SERCA1 proteins on SERCA1 and SERCA2.

One of the mechanisms involved in the lowering of the calcium in the ER in the cells expressing the truncated SERCA1s may be calcium leakage from the ER.

The amount of calcium leakage from the ER was measured after the plateau of calcium accumulation in the ER following addition of TbuBHQ (SERCA inhibitor). The level of calcium leakage was evaluated at various calcium concentrations using a function derived from the leakage slope. The authors of the invention obtained results which show an increase in the amount of calcium leakage in the cells expressing S1T+4 and S1T-4 compared to the control.

This result is compatible with the hypothesis that the SERCA1-T dimers might act as a cation pore.

This study suggests a model of control of calcium deposits and of regulation of the mechanisms of apoptosis in vitro by the X/SERCA1 hybrid proteins and the truncated SERCA proteins. This set of results indicates that the proteins might regulate the calcium stores of the ER by their ability to form dimers. Specifically, these dimers may contribute, by forming a pore, to calcium leakage from the ER to the cytoplasm.

The authors of the invention were also able to show that the truncated SERCA proteins have a dominant negative effect on both SERCA1 and SERCA2b.

All of our results indicate the mutation of an SERCA gene in human cancer and suggest a relationship between normal and truncated SERCA proteins, calcium homeostasis of the ER, apoptosis and cell transformation.

### Bibliography

Antonarakis S. E., N Engl J. Med. 320:153-163 (1989).

Bellis et al., medicine/sciences, 13:1317-24, (1997).

Berridge et al., Nature, 395(6703):645-8 (1998).

Benoit et al., PNAS USA, 79, 917-921 (1982).

Bik K Tye, Annu. Rev. Biochem. 68:649:686 (1999).

Burris, T. P. et al., Proc Natl Acad Sci USA, 92:9525-9 (1995).

Chami et al, Oncogene, 19:2877-2886 (2000).

Cooper et al., Diagnosis of genetic disease using recombinant DNA, 3^{rd} Edition, Hum Genet., 87:519-560 (1991).

Durfee, T et al., J Cell Biol, 127:609-22 (1994).

Dejean et al, Nature, 322:70-72 (1986).

Huber, C. G. et al., Anal. Chem, 67:578-585 (1995).

Ito et al, Molecular Cell, 3:361-370 (1999).

Kearsey et al, Biochim. Biophys. Acta, 1398(2):113-36 (1998).

Köhler and Milstein, Nature, 256, 495-497 (1975).

Kroes, R. A. et al., Cancer Lett, 156:191-8 (2000).

Kuklin, A. et al., Genetic Testing, 1:201-206 (1997/98).

Lin et al., Mol. Carcinogenesis, 26:53-61 (1999).

McCallum, C. et al., J Cell Biol, 149:591-602 (2000).

Minami et al, Genomics, 29:403-408 (1995).

Nielsen et al, Anticancer Drug Des., 8(1):53-63 (1993).

Paterlini et al, Primary liver cancer in HbsAg-negative patients: A study of HBV genome using the polymerase chain reaction. In “viral hepatitis and Liver Disease”, 556-559, Williams and Wilkins, Baltimore (1990).

Paterlini et al., Hepatitis B virus and primar liver cancer in hepatitis B surface antigen-positive and negative patients. In: Primary liver cancer, etiological and progression factors, London:B.C. 167-190 (1994).

Petretti, T. et al., Gut, 46:359-66 (2000).

Pineau et al., J. Virol. 70:7280-7284 (1996).

Poussin K et al., Int J Cancer, 80:497-505, (1999).

Pozzan et al., Physiol. Rev. 74(3):595-636 (1994).

Russo, G. et al., J Biol Chem, 272:5229-35 (1997).

Sambrook et al., Molecular cloning, a laboratory manual Spring Harbor Laboratory Press, 9.54-62 (1989).

Schneider, F. et al., Cancer Res, 61:4605-11 (2001).

Shibayama, E. & Koizumi, H., Am J Pathol 148:1807-18 (1996).

Sugimoto, T. et al., Jpn J Cancer Res, 92:152-60 (2001).

Tsuei et al, J. Virol. Methods, 49:269-284 (1994).

Urquidi et al., Annu. Rev. Med. 51:65-79 (2000).

Wang et al., Nature, 343:555-557 (1990).

Wang, Y. et al., Int J Oncol, 16:757-62 (2000).

Won, K. et al., Mol Cell Biol, 18:7584-7589 (1998).

Zhang et al, Biochem. Biophys. Res. Commun., 188:344-351 (1992).

Zhu, Y. et al., Biochim Biophys Acta, 1536:148-60 (2001).

Ziemiecki, A. et al.,). Embo J, 9:191-6 (1990).

1. An isolated nucleic acid comprising a nucleotide sequence selected from SEQ ID Nos. 2, 12, 14, 16, 18, 27, 29, 1, 3, 4, 5, 6, 7, 8, 9, 10 and 11 or a nucleotide sequence such that it encodes one of the amino acid sequences SEQ ID Nos. 13, 15, 17, 19, 28 and 30.

2. An isolated nucleic acid referred to as 83T, comprising a nucleotide sequence included between nucleotides 881787 and 812959 of the supercontig NT025651.

3. An isolated nucleic acid referred to as FR7, comprising a nucleotide sequence included between nucleotides 2128117 and 2130561 of the supercontig NT009622.

4. An isolated nucleic acid comprising a nucleotide sequence located in proximity to one of the sequences SEQ ID Nos. 7 and 10.

5. An isolated polypeptide comprising an amino acid sequence selected from SEQ ID Nos. 13, 15, 17, 19, 28 and 30, or a sequence encoded by one of the nucleotide sequences SEQ ID Nos. 2, 12, 14, 16, 18, 27, 29, 1, 3, 4, 5, 6, 7, 8, 9, 10 and 11 or by a nucleic acid as claimed in claim 2 or 3.

6. A cloning and/or expression vector comprising a nucleic acid sequence as claimed in claim 1, 2 or 3.

7. A host cell transfected with a vector as claimed in claim 6.

8. A method of producing a polypeptide as claimed in claim 5, in which an expression vector as claimed in claim 6 is transferred [lacuna].

9. The use of a nucleic acid as claimed in claim 1, 2 or 3, for obtaining probes or primers having at least 15 nucleotides, which hybridize specifically with one of the nucleic acid sequences of claim 1, or the sequence complementary thereto, under stringent hybridization conditions.

10. An antibody directed against the polypeptide as defined in claim 5.

11. The use of at least one antibody as claimed in claim 10, for detecting or purifying a polypeptide as defined in claim 5 in a biological sample.

12. A kit comprising:
at least one antibody as claimed in claim 10, optionally attached to a support;
means of revealing the formation of specific antigen/antibody complexes between the polypeptide of claim 5 and said antibody, and/or means of quantifying these complexes.

13. A method of in vitro diagnosis of a tumor or of a predisposition to developing a tumor, comprising the steps consisting in:
a1) bringing a biological sample containing DNA or RNA into contact with specific oligonucleotides allowing the amplification of all or part of a gene involved in oncogenesis or of its transcript, said gene comprising a sequence selected from SEQ ID Nos. 1, 3, 7, 9, 10, 11, 20, 24, 2, 12, 14, 16, 18, 21, 25, 27 and 29;
b1) amplifying said DNA or RNA;
c1) detecting the amplification products;
d1) comparing the amplification products obtained to those obtained with a control sample, and detecting in this way a possible abnormality in said gene or in its transcript or else an abnormal number of copies of said gene, indicating a tumor or a predisposition to developing a tumor, or
a2) bringing a biological sample containing mRNA obtained from a sample of suspect cells from a patient into contact with specific oligonucleotides allowing the amplification of all or part of the transcript of said gene, comprising a sequence selected from SEQ ID Nos. 1, 3, 7, 9, 10, 11, 20, 24, 2, 12, 14, 16, 18, 21, 25, 27 and 29;
b2) amplifying said transcript;
c2) detecting and quantifying the amplification products;
a modification of the level of transcript of said gene compared to the normal control being an indicator of a tumor or of a predisposition to developing a tumor.

14. A method of in vitro diagnosis of a tumor or of a predisposition to developing a tumor, comprising the steps consisting in:
a1) bringing a biological sample containing DNA or RNA into contact with specific oligonucleotides allowing the amplification of all or part of a gene involved in oncogenesis or of its transcript, said gene being selected from IP₃R₁, CCT7, NMP p84, TRKB, TRUP and ST3GalVI;
b1) amplifying said DNA or RNA;
c1) detecting the amplification products;
d1) comparing the amplification products obtained to those obtained with a control sample, and detecting in this way a possible abnormality in said gene or in its transcript or else an abnormal number of copies of said gene, indicating a predisposition to developing a tumor, or
a2) bringing a biological sample containing mRNA obtained from a sample of suspect cells from a patient into contact with specific oligonucleotides allowing the amplification of all or part of the transcript of said gene selected from IP₃R₁, CCT7, NMP p84, TRKB, TRUP and ST3GalVI;
b2) amplifying said transcript;
c2) detecting and quantifying the amplification products;
the modification of the level of transcript of said gene compared to the normal control being an indicator of a tumor or of a predisposition to developing a tumor.

15. A method of in vitro diagnosis of a tumor or of a predisposition to developing a tumor, comprising the steps consisting in:
a1) bringing a biological sample containing DNA or RNA into contact with specific oligonucleotides allowing the amplification of all or part of a gene involved in oncogenesis or of its transcript, said gene comprising an isolated nucleic acid as claimed in either of claims 2 and 3;
b1) amplifying said DNA or RNA;
c1) detecting the amplification products;
d1) comparing the amplification products obtained to those obtained with a control sample, and detecting in this way a possible abnormality in said gene or in its transcript or else an abnormal number of copies of said gene, indicating a predisposition to developing a tumor, or
a2) bringing a biological sample containing mRNA obtained from a sample of suspect cells from a patient into contact with specific oligonucleotides allowing the amplification of all or part of the transcript of said gene, comprising an isolated nucleic acid as claimed in either of claims 2 and 3;
b2) amplifying said transcript;
c2) detecting and quantifying the amplification products;
the modification of the level of transcript of said gene compared to the normal control being an indicator of a tumor or of a predisposition to developing a tumor.

16. A method of in vitro diagnosis of a tumor or of a predisposition to developing a tumor, comprising bringing at least one antibody directed against the polypeptide comprising a sequence selected from SEQ ID Nos. 13, 15, 17, 19, 22, 26, 28 or 30 or encoded by a nucleotide sequence comprising a sequence selected from SEQ ID Nos. 1, 3, 7, 9, 10, 11, 20, 24, 2, 12, 14, 16, 18, 21, 25, 27 and 29 into contact with a biological sample, under conditions which allow the possible formation of specific immunocomplexes between said polypeptide and said antibody or antibodies, and detecting and/or quantifying the specific immunocomplexes possibly formed.

17. A method of in vitro diagnosis of a tumor or of a predisposition to developing a tumor, comprising bringing at least one antibody directed against a polypeptide selected from IP₃R₁, CCT7, NMP p84, TRKB, TRUP and ST3GalVI into contact with a biological sample, under conditions which allow the possible formation of specific immunocomplexes between said polypeptide and said antibody or antibodies, and detecting and/or quantifying the specific immunocomplexes possibly formed.

18. A method of in vitro diagnosis of a tumor or of a predisposition to developing a tumor, comprising bringing at least one antibody directed against the polypeptide encoded by an isolated nucleic acid as claimed in either of claims 2 and 3 into contact with a biological sample, under conditions which allow the possible formation of specific immunocomplexes between said polypeptide and said antibody or antibodies, and detecting and/or quantifying the specific immunocomplexes possibly formed.

19. A pharmaceutical composition comprising a nucleic acid as claimed in one of claims 1, 2 and 3 or a polypeptide as claimed in claim 5, in combination with a pharmaceutically acceptable vehicle.

20. A pharmaceutical composition comprising a nucleic acid which is an antisense of the nucleic acid as claimed in claim 1, 2 or 3, or an antibody as claimed in claim 10, in combination with a pharmaceutically acceptable vehicle.

21. The use of a nucleic acid comprising a sequence selected from SEQ ID Nos. 1, 3, 7, 9, 10, 11, 20, 24, 2, 12, 14, 16, 18, 21, 25, 27 and 29, of an antisense of said nucleic acid, of a polypeptide encoded by said nucleic acid, or of an antibody against said polypeptide, for producing a medicinal product intended to treat tumors.

22. The use of a nucleic acid comprising a sequence of a gene selected from IP₃R₁, CCT7, NMP p84, TRKB, TRUP and ST3GalVI, of an antisense of said nucleic acid, of a polypeptide encoded by said nucleic acid, or of an antibody against said polypeptide, for producing a medicinal product intended to treat tumors.

23. The use of a nucleic acid as claimed in either of claims 2 and 3, of an antisense of said nucleic acid, of a polypeptide encoded by said nucleic acid, or of an antibody against said polypeptide, for producing a medicinal product intended to treat tumors.

24. A method of detecting genes involved in oncogenesis, comprising the steps consisting in
extracting DNA from a tumoral liver tissue;
amplifying, in vitro, by Alu-PCR, a nucleotide sequence at the viral DNA/cellular DNA junction;
sequencing said nucleotide sequence,
identifying one or more genes comprising, or located in proximity to, said sequence thus sequenced;
characterized in that the amplification step uses a primer comprising the sequence 5′TGCCCAAGGTCTTACATAAGAGGA3′.

25. The use of a gene identified using the method as claimed in claim 24, for the in vitro diagnosis of a tumor or of a predisposition to developing a tumor.

26. The use of a gene identified using the method as claimed in claim 24, of an antisense of said gene, of a polypeptide encoded by said gene, or of an antibody against the said polypeptide, for producing a medicinal product intended to treat tumors.


SEQUENCE LISTING (30 sequences)

SEQ ID NO: 1 - DNA, 660 bp, Homo sapiens
cttctggtataaagtggggaagattacacttatgtgatcaccaaaggatttactagtatcttggtcattccaattgcacaatgttaactgtacaacacacagcagaaaagtgaatagacttcactaagggattctaagtttagaaaataggttttgttttcttaaaaaattttgtgtataatacaaactaatgaaaactatacatattctccaattcctatagtaataataatgtaactgttacaccaactttcctcatatttgagagatgagtacatgttggattgcagcatttcttcatgttaaaaacatggaatattattcaaatatagtacttggggcctaaacaactaaaattagtcaccgcataactagttgaaaatggcataggcataaaatgttaataaagaatggcagttatatttatgctcacttcctggagtaattgggtaatattcagaaaggcacatcgtggtagtttaagtgtacaaggctttagggcagtatctagcccagtattattccagatactcctgagctctatataaagggcattttggaggaactcttctgggcaatatctttcccaatctcatctaaattctgggaaatattttccatagcagacaacccagctctgaaactaagcacctggtgatc

SEQ ID NO: 2 - DNA, 190 bp, Homo sapiens
cgtggtagtttaagtgtacaaggctttagggcagtatctagcccagtattattccagatactcctgagctctatataaagggcattttggaggaactcttctgggcaatatctttcccaatctcatctaaattctgggaaatattttccatagcagacaacccagctctgaaactaagcacctggtgatc

SEQ ID NO: 3 - DNA, 475 bp, Homo sapiens
  MISC_FEATURE (361)...(361); (410)...(410); (434)...(434): n = A or C or G or T
tttaaaatatgactttgttatgcttttgtgtaatcatgactatacactgttgtaaaaataatgagagttctcatttctagaggatctaaaatttgtaatggtctttctgttatagtcagtattaatttctattactttggctaataagaaggttgggtgacttcttattctatctctgctctttatcttatcaaataactagtctcctctgctaagcctttcagactttgcctctcttcaagattaggctccaaattcagagcctactttggcagtacattttctgcaccctgtctttggtctttcctagagggctgctacttgggtagcatcaatcactttactttcttatcttgcttgnataaccatggcttgactcacacacagaggttattctaaaaattacaatnggcagaagatgctctaagttttgnaatatgctcaatgatattttaaaagaactggtctatccggc

SEQ ID NO: 4 - DNA, 520 bp, Homo sapiens
  MISC_FEATURE (180) ... (180): n = A or C or G or T
ggaaatactcaagaagaaaaacctaggagactttgcaagaaatatttaaataaaggtgattaaatacattggtaacatattctgttgtctaaacaacaacagcaggaacagcagcagacttaagaccaaagacaaagggaaagcaaacccataagaatcccaaattaaaattctggacantattacattgatagtgggttgtgagtatctagaaatttgggaaatccccaaatatttggaaattagcatatttttacattatccatggatcaaagaagaaatcacaaggaaaattattaaatatcttttgaactaaatgaaaaagaaaatgcaacgtatcaaaatgtatgggatgcggcaaatgcagcatttaagggaactgtatagctataaatacctagattagaaaagaagaatggtctcatatcaaccaatctaagcttctactttaagcaactggaaaaagaaggaacaattaaatctgaagcaaacagcagaaagaaaataaagattagagtgg

SEQ ID NO: 5 - DNA, 245 bp, Homo sapiens
tctttgcagccatttaaataagcttatgatatctcttatgtgatctccctcaactaagatgtcaacttcttgttaaccacgttatctctaccatttctaaaggaaaaatccttttcctcgccagagaaagacacagaataagaggtagaaaatactgctttgtctctattatctgttaatgtctcaccatctgcccaaactctggtcttgtcacttcatccttcttactctaagcctggttcaaa

SEQ ID NO: 6 - DNA, 648 bp, Homo sapiens
taagtaaatatgcttcagatggccctgagtatctggcagcacaaagtgcccagggaaaaagcattgcaatagccctttccatccctaacatcaaatgctgctttccagccagaaagcttatttccaagtgttcactggtccaaaaatctacattattctggatttcaatcatgtagtttagttcaaactatgagcaactgctttccagttactctgctcagcaagtattttctttccctctggttggcatatgaggcctgttgtaaccatggccatcattcaaatgatgcattttcttctcccaactcctcccaagcccagagaaggttaggaattccagagaaccactagtcccaaacagtatctctctgccaggcttaatcttctgatccaagcaaaaaaagtaaaattgacaagtttcttctttccctggaggccagactcatggtccaaaaataatctgactatcttggcatctggtttgtccagtttcagtcatactccttgtcttgtgggacttctatttgcaatgggatatttcagtagatgaaaattaatactttatatcacagattgaggtctgaaggcctcaaatatttagtactaactagaaatgccagtttcaaatatatctgtgtttctggccag

SEQ ID NO: 7 - DNA, 1059 bp, Homo sapiens
gttgcctcaatgcaactctgctgacataatatatttcataaacatagaaatcctgtatgtcttgtctgttttcttttatatctctcatacctggcatagtgcctgaaaaatagaaaatgttcagtaaatatgcttttcaacgaatgaggaggaagaaatggagagacacataaagcatgatggatgttttacatatctaactactgatcaaaatgtggaataaattactttatgcccaactaatcatgcatataccacgtctgtatttcattgatgttgtactgacattattagcaataggtggcagtcagtaaataatccaagccatatcaaccataggaacacttatcagatttttgtttggcttatctgtctttgctgtcttgcatccatttcccctccttaatggcatcacaatttcctagtttcattgtgtacagtctcaatggcccagtaactccagattcctctctccaactacagaagccaaggagggcttgacagaatctacttctcctaccctgtgatagagccaggaggcaagcaactggcctatgcatggccaatcaaatgatctccctctaagacttcgaattttgtataagtgaaacaaggatggaaagaacgtttggcatttactcatcacagtggttaagagctgtatcaaaaccagactgctcctgctatgggacagttactgtgtttccaacttcctgtacccgtaggcatctcagtttttgcccatttccactattgaatctcagagctcatgaaatccttctgcttgaattagtaagagatggtttctgttgtttaccaccaaggagctctaagtggcacatgttcacaccagcatcatagcagccagtctgcatccttggaagatgctaccatgtgatgtctagcaagagtacagaaacaaccgtaatacacaactcccaggaggaaagagggaaaagcagggttgtattcttcaatattgtcaattctgctttttaaagttaagtatagctatgcctggcttaaacaataatgtatacaattaatcacatttgtttc

SEQ ID NO: 8 - DNA, 174 bp, Homo sapiens
agttttgtcaaagtttcaggggtcttcattgctcagtctcttttaccacaggctggaaggtgaggggaggtgggatggtgtgactggggataagttcatcccaaactcctgaacatgtagaacatctgtacagtggaaaccgtaaatagggcaactgacagaaaataattgcaa

SEQ ID NO: 9 - DNA, 59 bp, Homo sapiens
tgtttattttgagcctatgtatgtctttgcacatgagatggatctcctaaatacagcac

SEQ ID NO: 10 - DNA, 214 bp, Homo sapiens
atattgcaatgtcggacccattttctttactctccctacagcgtgcactgtacagtagttgcccaktaaactatgatttgaaatgcaaaacgaaatcaaattaggtagagcacgtaagatgactcactgcatgtacaattttaggatgtcccagaatatgtatgaaacaatgtgatcaaattacaatgtactgataactggcattaaattcatt

SEQ ID NO: 11 - DNA, 457 bp, Homo sapiens
aaatgttttctgtattctgatttgccttctttaccaagataaataaagaagcagttaaagaacgtattacctttgtgggaagactgtattttccatctggaagaggaaagctctgaatttctccacatgcagcacaaagaaaagccatcttggtgcaaagaggctttatattactgacacgaaccactgtccctcttagagcaatgtattttccatagtaatttgctctgacattcttgagctgtgtcaaaggctcatagttgtacaccctaaagacaaaacaggaagtaatgaacagaaaattgatgttttttatattttcacaagttccttttaggagcaactatggaattctcaaatcaaatcatcccttcgatatatcagaaatatcacaagaattaaaccattcttgttgtgttttgttttcttgagatggagtctcgctctgtcgcccggg

SEQ ID NO: 12 - DNA, 2523 bp, Homo sapiens
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtccgagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaaggttctcgaaataagaatgacaagtgtatgttccttttgtatattgaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaacttgttaaagcaggtttggcattagcactctttggaggaagccagaaatacgcagatgacaaaaacagaattccaattcggggagacccccacatccttgttgttggagatccaggcctaggaaaaagtcagatgctacaggcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaa

SEQ ID NO: 13 - protein, 841 aa, Homo sapiens
MNGEYRGRGFGRGRFQSWKRGRGGGNFSGKWREREHRPDLSKTTGKRTSEQTPQFLLSTKTPQSMQSTLDRFIPYKGWKLYFSEVYSDSSPLIEKIQAFEKFFTRHIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEGSRNKNDKCMFLLYIEANSISNSKGQKTKSSEDGCKHGMLMEFSLKDLYAIQEIQAEENLFKLIVNSLCPVIFGHELVKAGLALALFGGSQKYADDKNRIPIRGDPHILVVGDPGLGKSQMLQAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKMGSALLSRFDLVFILLDTPNEHHDHLLSEHVIAIRAGKQRTISSATVARMNSQDSNTSVLEVVSEKPLSERLKVVPGETIDPIPHQLLRKYIGYARQYVYPRLSTEAARVLQDFYLELRKQSQRLNSSPITTRQLESLIRLTEARARLELREEATKEDAEDIVEIMKYSMLGTYSDEFGNLDFERSQHGSGMSNRSTAKRFISALNNVAERTYNNIFQFHQLRQIAKELNIQVADFENFIGSLNDQGYLLKKGPKVYQLQTMZ

SEQ ID NO: 14 - DNA, 2475 bp, Homo sapiens
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtccgagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaacttgttaaagcaggtttggcattagcactctttggaggaagccagaaatacgcagatgacaaaaacagaattccaattcggggagacccccacatccttgttgttggagatccaggcctaggaaaaagtcagatgctacaggcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaa

SEQ ID NO: 15 - protein, 825 aa, Homo sapiens
MNGEYRGRGFGRGRFQSWKRGRGGGNFSGKWREREHRPDLSKTTGKRTSEQTPQFLLSTKTPQSMQSTLDRFIPYKGWKLYFSEVYSDSSPLIEKIQAFEKFFTRHIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEANSISNSKGQKTKSSEDGCKHGMLMEFSLKDLYAIQEIQAEENLFKLIVNSLCPVIFGHELVKAGLALALFGGSQKYADDKNRIPIRGDPHILVVGDPGLGKSQMLQAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKMGSALLSRFDLVFILLDTPNEHHDHLLSEHVIAIRAGKQRTISSATVARMNSQDSNTSVLEVVSEKPLSERLKVVPGETIDPIPHQLLRKYIGYARQYVYPRLSTEAARVLQDFYLELRKQSQRLNSSPITTRQLESLIRLTEARARLELREEATKEDAEDIVEIMKYSMLGTYSDEFGNLDFERSQHGSGMSNRSTAKRFISALNNVAERTYNNIFQFHQLRQIAKELNIQVADFENFIGSLNDQGYLLKKGPKVYQLQTMZ

SEQ ID NO: 16 - DNA, 2382 bp, Homo sapiens
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtccgagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaaggttctcgaaataagaatgacaagtgtatgttccttttgtatattgaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaagcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaa

SEQ ID NO: 17 - protein, 794 aa, Homo sapiens
MNGEYRGRGFGRGRFQSWKRGRGGGNFSGKWREREHRPDLSKTTGKRTSEQTPQFLLSTKTPQSMQSTLDRFIPYKGWKLYFSEVYSDSSPLIEKIQAFEKFFTRHIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEGSRNKNDKCMFLLYIEANSISNSKGQKTKSSEDGCKHGMLMEFSLKDLYAIQEIQAEENLFKLIVNSLCPVIFGHEAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKMGSALLSRFDLVFILLDTPNEHHDHLLSEHVIAIRAGKQRTISSATVARMNSQDSNTSVLEVVSEKPLSERLKVVPGETIDPIPHQLLRKYIGYARQYVYPRLSTEAARVLQDFYLELRKQSQRLNSSPITTRQLESLIRLTEARARLELREEATKEDAEDIVEIMKYSMLGTYSDEFGNLDFERSQHGSGMSNRSTAKRFISALNNVAERTYNNIFQFHQLRQIAKELNIQVADFENFIGSLNDQGYLLKKGPKVYQLQTMZ

SEQ ID NO: 18 - DNA, 2332 bp, Homo sapiens
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtccgagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaaggttctcgaaataagaatgacaagtgtatgttccttttgtatattgaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaagcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaa

SEQ ID NO: 19 - protein, 535 aa, Homo sapiens
MNGEYRGRGFGRGRFQSWKRGRGGGNFSGKWREREHRPDLSKTTGKRTSEQTPQFLLSTKTPQSMQSTLDRFIPYKGWKLYFSEVYSDSSPLIEKIQAFEKFFTRHIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEGSRNKNDKCMFLLYIEANSISNSKGQKTKSSEDGCKHGMLMEFSLKDLYAIQEIQAEENLFKLIVNSLCPVIFGHEAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKYSKZ

SEQ ID NO: 20 - DNA, 258 bp, Homo sapiens
cctggcccataatgtgtttcttttaatcccaacagagcatcactttgggtcctcaggaatgacattacatgaacgctttactaaatacctaaagagaggaactgagcaggaggcagccaaaaacaagaaaagcccagagatacacaggtaaggaccatggccttatactggaggtgtaataaaagactttgtatcagacattaaactcaccttgttaaattctgccgctaatgcacctttaatacaaaatttacagta

SEQ ID NO: 21 - DNA, 3618 bp, Homo sapiens
ggcacgagcgaggttcgggctggttgttccgttgcgagctgcagctgcgatctctgtggtaggcccagaagtgtatgctgacttgtaaagtgaagaagccagtggtgctgcgggtgttcttttggggtagtgtctgggatccagtacgagttgaatcattgttcaaataaggtgtaattgaaaagtgatcctctcttcagagatgtcaaaaacaaacaaatccaagtctggatctcgctcttctcgctcaagatctgcatcaagatctcgttctcgttcattttcgaagtctcggtcccgaagccgatctctctctcgttcaaggaagcgcaggctgagttctaggtctcgttccagatcatattctccagctcataacagagaaagaaaccacccaagagtatatcagaatcgggatttccgaggtcacaacagaggctatagaaggccctattatttccgtgggcgtaacagaggcttttatccatggggccaatataaccgaggaggctatggaaactaccgctcaaattggcagaattaccggcaagcatacagtcctcgtcgaggccgttcaagatcccggtccccaaagagaaggtccccttcaccaaggtccaggagccattctagaaactctgataagtcgtcttctgaccggtcaaggcgctcctcatcctcccgttcttcctccaaccatagccgagttgaatcttctaagcgcaagtctgcaaaggagaaaaagtcctcttctaaggatagccggccatctcaggctgccggggataaccagggagatgaggtcaaggagcagacattctctggaggcacctctcaagatacaaaagcatctgagagctcgaagccatggccagatgccacctacggcactggttctgcatcacgggcctcagcagtttctgagctgagtcctcgggagcgaagcccagctctcaaaagccccctccagtctgtggtggtgaggcggcggtcaccccgtcctagccccgtgccaaaacctagtcctccactttccagcacatcccagatgggctcaactctgccgagtggtgccgggtatcagtctgggacacaccaaggtcagttcgaccatggttctgggtccctgagtccatccaaaaagagccctgtgggtaagagtccaccatccactggctccacatatggctcatctcagaaggaggagagtgctgcttcaggaggagcagcctatacaaagaggtatctagaagagcagaagacagagaatggaaaagataaggaacagaaacaaacaaataccgataaagaaaaaataaaagagaaagggagcttctctgacacaggcttgggtgatggaaaaatgaaatctgattcttttgctcccaaaactgattctgagaagccttttcggggcagtcagtctcccaaaaggtataagctccgagatgactttgagaagaagatggctgacttccacaaggaggagatggatgatcaagataaggacaaagctaagggaagaaaggaatctgagtttgatgatgaacccaaatttatgtctaaagtcataggtgcaaacaaaaaccaggaggaggagaagtcaggcaaatgggagggcctggtatatgcacctccagggaaggaaaagcagagaaaaacagaggagctggaggaggagtctttcccagagagatccaaaaaggaagatcggggcaagagaagcgaaggtgggcacaggggctttgtgcctgagaagaatttccgagtgactgcttataaagcagtccaggagaaaagctcatcacctcccccaagaaagacctctgagagccgagacaagctgggagcgaaaggagattttcccacaggaaagtcttccttttccattactcgagaggcacaggtcaatgtccggatggactcttttgatgaggacctcgcacgacccagtggcttattggctcaggaacgcaagctttgccgagatctagtccatagcaacaaaaaggaacaggagtttcgttccattttccagcacatacaatcagctcagtctcagcgtagcccctcagaactgtttgcccaacatatagtgaccattgttcaccatgttaaagagcatcactttgggtcctcaggaatgacattacatgaacgctttactaaatacctaaagagaggaactgagcaggaggcagccaaaaacaagaaaagcccagagatacacaggagaatagacatttcccccagtacattcagaaaacatggtttggctcatgatgaaatgaaaagtccccgggaacctggctacaaggctgagggaaaatacaaagatgatcctgttgatctccgccttgatattgaacgtcgtaaaaaacataaggagagagatcttaaacgaggtaaatcgagagaatcagtggattcccgagactccagtcactcaagggaaaggtcagctgaaaaaacagagaaaactcataaaggatcaaagaaacagaagaagcatcggagagcaagagacaggtccagatcctcctcctcttcctcccagtcatctcactcctacaaagcagaagagtacactgaagagacagaggaaagagaggagagcaccacgggctttgacaaatcaagactggggaccaaagactttgtgggtccaagtgaaagaggaggtggcagagctcgaggaacctttcagtttcgagccagaggaagaggctggggcagaggcaactactctgggaacaataacaacaacagcaacaacgattttcaaaaaagaaaccgggaagaggagtgggacccagagtacacacccaaaagcaagaagtattacttgcatgatgaccgtgaaggcgaaggcagtgacaagtgggtgagccggggccggggccgaggagcctttcctcggggtcggggccggttcatgttccggaaatcaagtaccagccccaagtgggcccatgacaagttcagtggggaggaaggggagattgaagacgacgagagtgggacagagaaccgagaagagaaggacaatatacagcccacaaccgagtaggggccacccttgacgggattcctgcccaggggagagaggcgctgggaagatggctggtgaggagcttaacagaggaacctcaagaagattctgaaaatcctacccccaccccccaccagccgcacagattgtactaccgcgagaggcatccctggcgctgtctcccactggacagaggaggctggccatggggcccaggggtcaggcccagcttttgagcagaatacaacgcattgggctttagctgtttttctcatttgttggtgtgtggggtgggggcaggggtagggcgggagagcgatgcttggatttttgtttcctattagaaaccaacagttttgttctaatttcatttcatttggagctaagatgactaatttgatgattttcgatctcttttcccctgtcctgattttaaaagccccctccttttttttttttttttttctttttttaggcatatgtagtaatattagaaacatttaatttgggaaactttgattcttgaaagagaaaacaaaagcatgtgaataaactttgctcgtgcc

SEQ ID NO: 22 - protein, 38 aa, Homo sapiens
EHHFGSSGMTLHERFTKYLKRGTEQEAAKNKKSPEIHR

SEQ ID NO: 23 - DNA, 238 bp, Homo sapiens
tctgtgttggtcaggctggtctcgaactcctgacctcaggcgatctgcccgccttggcctcccaaagtgctgggattacaggcatgagccactgcgcctggcttttctacttttttgatgtagctgcttatagctataaacttccttcttagcactgcttttgctgtataccgtgggttttgagaggttatgtttccgttatcatttgcttaaataaatttttcaatttcctttttaa

SEQ ID NO: 24 - DNA, 289 bp, Homo sapiens
gctggtgatacagcagtttgaagacctcctggtgcggattctcctcctggccgcatgcatttccttcgtaagtgtgggagggtctctgcgggctggctgggggtgtgaggctgggatcgggcgaatgcggggctcgcagtcactggatcctcccgtccgagtcccgagcatcccattgtacagacggggcgggctggcgcgcagcagcgggtgtgattcgcgtcctcctctctcctcccctgcaccccagaggcaggttttattttaagctttaagggtgttctcagca

SEQ ID NO: 25 - DNA, 3309 bp, Homo sapiens
  CDS (1)..(2982)
atggaggccgctcatgctaaaaccacggaggaatgtttggcctattttggggtgagtgagaccacgggcctcaccccggaccaagttaagcggaatctggagaaatacggcctcaatgagctccctgctgaggaagggaagaccctgtgggagctggtgatagagcagtttgaagacctcctggtgcggattctcctcctggccgcatgcatttccttcgtgctggcctggtttgaggaaggtgaagagaccatcactgcctttgttgaaccctttgtcatcctcttgatcctcattgccaatgccatcgtgggggtttggcaggagcggaacgcagagaacgccatcgaggccctgaaggagtatgagccagagatggggaaggtctaccgggctgaccgcaagtcagtgcaaaggatcaaggctcgggacatcgtccctggggacatcgtggaggtggctgtgggggacaaagtccctgcagacatccgaatcctcgccatcaaatccaccacgctgcgggttgaccagtccatcctgacaggcgagtctgtatctgtcatcaaacacacggagcccgttcctgacccccgagctgtcaaccaggacaagaagaacatgcttttctcgggcaccaacattgcagccggcaaggccttgggcatcgtggccaccaccggtgtgggcaccgagattgggaagatccgagaccaaatggctgccacagaacaggacaagacccccttgcagcagaagctggatgagtttggggagcagctctccaaggtcatctccctcatctgtgtggctgtctggcttatcaacattggccacttcaacgaccccgtccatgggggctcctggttccgcggggccatctactactttaagattgccgtggccttggctgtggctgccatccccgaaggtcttcctgcagtcatcaccacctgcctggccctgggtacccgtcggatggcaaagaagaatgccattgtaagaagcttgccctccgtagagaccctgggctgcacctctgtcatctgttccgacaagacaggcaccctcaccaccaaccagatgtctgtctgcaagatgtttatcattgacaaggtggatggggacatctgcctcctgaatgagttctccatcaccggctccacttacgctccagagggagaggtcttgaagaatgataagccagtccggccagggcagtatgacgggctggtggagctggccaccatctgtgccctctgcaatgactcctccttggacttcaacgaggccaaaggtgtctatgagaaggtcggcgaggccaccgagacagcactcaccaccctggtggagaagatgaatgtgttcaacacggatgtgagaagcctctcgaaggtggagagagccaacgcctgcaactcggtgatccgccagctaatgaagaaggaattcaccctggagttctcccgagacagaaagtccatgtctgtctattgctccccagccaaatcttcccgggctgctgtgggcaacaagatgtttgtcaagggtgcccctgagggcgtcatcgaccgctgtaactatgtgcgagttggcaccacccgggtgccactgacggggccggtgaaggaaaagatcatggcggtgatcaaggagtggggcactggccgggacaccctgcgctgcttggccctggccacccgggacacccccccgaagcgagaggaaatggtcctggatgactctgccaggttcctggagtatgagacggacctgacattcgtgggtgtagtgggcatgctggaccctccgcgcaaggaggtcacgggctccatccagctgtgccgtgacgccgggatccgggtgatcatgatcactggggacaacaagggcacagccattgccatctgccggcgaattggcatctttggggagaacgaggaggtggccgatcgcgcctacacgggccgagagttcgacgacctgcccctggctgaacagcgggaagcctgccgacgtgcctgctgcttcgcccgtgtggagccctcgcacaagtccaagattgtggagtacctgcagtcctacgatgagatcacagccatgacaggtgatggcgtcaatgacgcccctgccctgaagaaggctgagattggcattgccatgggatctggcactgccgtggccaagactgcctctgagatggtgctggctgacgacaacttctccaccatcgtagctgctgtggaggagggccgcgccatctacaacaacatgaagcagttcatccgctacctcatttcctccaacgtgggcgaggtggtctgtatcttcctgaccgctgccctggggctgcctgaggccctgatcccggtgcagctgctatgggtgaacttggtgaccgacgggctcccagccacagccctgggcttcaacccaccagacctggacatcatggaccgccccccccggagccccaaggagcccctcatcagtggctggctcttcttccgctacatggcaatcgggggctatgtgggtgcagccaccgtgggagcagctgcctggtggttcctgtacgctgaggatgggcctcatgtcaactacagccagctgactcacttcatgcagtgcaccgaggacaacacccactttgagggcatagactgtgaggtcttcgaggcccccgagcccatgaccatggccctgtccgtgctggtgaccatcgagatgtgcaatgcactgaacagcctgtccgagaaccagtccctgctgcggatgccaccctgggtgaacatctggctgctgggctccatctgcctctccatgtccctgcacttcctcatcctctatgttgaccccctgccgatgatcttcaagctccgggccctggacctcacccagtggctcatggtcctcaagatctcactgccagtcattgggctcgacgaaatcctcaagttcgttgctcggaactacctagagggataactgttccccctcctccatctctgagcccgtgtcacagatccagaagatgaaagaaggaagtgagcatccttttgctctgtcctccccaccccgatagtgacacatcttcaggcagagctgtggcacagacccccgtcctgtcccccacacccgtgtcatgtgtctgttttataaacatgtccccttccctttccttccccctcggccacccgcctccctctcaaccttgtaaattccccttcccaaccccgaggggcttgcagggacaaggcgaccgactgcgctgagctgcttatttattgaaaataaacgacggaaaagtca

SEQ ID NO: 26 - protein, 994 aa, Homo sapiens
MEAAHAKTTEECLAYFGVSETTGLTPDQVKRNLEKYGLNELPAEEGKTLWELVIEQFEDLLVRILLLAACISFVLAWFEEGEETITAFVEPFVILLILIANAIVGVWQERNAENAIEALKEYEPEMGKVYRADRKSVQRIKARDIVPGDIVEVAVGDKVPADIRILAIKSTTLRVDQSILTGESVSVIKHTEPVPDPRAVNQDKKNMLFSGTNIAAGKALGIVATTGVGTEIGKIRDQMAATEQDKTPLQQKLDEFGEQLSKVISLICVAVWLINIGHFNDPVHGGSWFRGAIYYFKIAVALAVAAIPEGLPAVITTCLALGTRRMAKKNAIVRSLPSVETLGCTSVICSDKTGTLTTNQMSVCKMFIIDKVDGDICLLNEFSITGSTYAPEGEVLKNDKPVRPGQYDGLVELATICALCNDSSLDFNEAKGVYEKVGEATETALTTLVEKMNVFNTDVRSLSKVERANACNSVIRQLMKKEFTLEFSRDRKSMSVYCSPAKSSRAAVGNKMFVKGAPEGVIDRCNYVRVGTTRVPLTGPVKEKIMAVIKEWGTGRDTLRCLALATRDTPPKREEMVLDDSARFLEYETDLTFVGVVGMLDPPRKEVTGSIQLCRDAGIRVIMITGDNKGTAIAICRRIGIFGENEEVADRAYTGREFDDLPLAEQREACRRACCFARVEPSHKSKIVEYLQSYDEITAMTGDGVNDAPALKKAEIGIAMGSGTAVAKTASEMVLADDNFSTIVAAVEEGRAIYNNMKQFIRYLISSNVGEVVCIFLTAALGLPEALIPVQLLWVNLVTDGLPATALGFNPPDLDIMDRPPRSPKEPLISGWLFFRYMAIGGYVGAATVGAAAWWFLYAEDGPHVNYSQLTHFMQCTEDNTHFEGIDCEVFEAPEPMTMALSVLVTIEMCNALNSLSENQSLLRMPPWVNIWLLGSICLSMSLHFLILYVDPLPMIFKLRALDLTQWLMVLKISLPVIGLDEILKFVARNYLEG

SEQ ID NO: 27 - DNA, 3206 bp, Homo sapiens
  CDS (1)..(1251)
atggaggccgctcatgctaaaaccacggaggaatgtttggcctattttggggtgagtgagaccacgggcctcaccccggaccaagttaagcggaatctggagaaatacggcctcaatgagctccctgctgaggaagggaagaccctgtgggagctggtgatagagcagtttgaagacctcctggtgcggattctcctcctggccgcatgcatttccttcgtgctggcctggtttgaggaaggtgaagagaccatcactgcctttgttgaaccctttgtcatcctcttgatcctcattgccaatgccatcgtgggggtttggcaggagcggaacgcagagaacgccatcgaggccctgaaggagtatgagccagagatggggaaggtctaccgggctgaccgcaagtcagtgcaaaggatcaaggctcgggacatcgtccctggggacatcgtggaggtggctgtgggggacaaagtccctgcagacatccgaatcctcgccatcaaatccaccacgctgcgggttgaccagtccatcctgacaggcgagtctgtatctgtcatcaaacacacggagcccgttcctgacccccgagctgtcaaccaggacaagaagaacatgcttttctcgggcaccaacattgcagccggcaaggccttgggcatcgtggccaccaccggtgtgggcaccgagattgggaagatccgagaccaaatggctgccacagaacaggacaagacccccttgcagcagaagctggatgagtttggggagcagctctccaaggtcatctccctcatctgtgtggctgtctggcttatcaacattggccacttcaacgaccccgtccatgggggctcctggttccgcggggccatctactactttaagattgccgtggccttggctgtggctgccatccccgaaggtcttcctgcagtcatcaccacctgcctggccctgggtacccgtcggatggcaaagaagaatgccattgtaagaagcttgccctccgtagagaccctgggctgcacctctgtcatctgttccgacaagacaggcaccctcaccaccaaccagatgtctgtctgcaagatgtttatcattgacaaggtggatggggacatctgcctcctgaatgagttctccatcaccggctccacttacgctccagagggagaggtgccaaaggtgtctatgagaaggtcggcgaggccaccgagacagcactcaccaccctggtggagaagatgaatgtgttcaacacggatgtgagaagcctctcgaaggtggagagagccaacgcctgcaactcggtgatccgccagctaatgaagaaggaattcaccctggagttctcccgagacagaaagtccatgtctgtctattgctccccagccaaatcttcccgggctgctgtgggcaacaagatgtttgtcaagggtgcccctgagggcgtcatcgaccgctgtaactatgtgcgagttggcaccacccgggtgccactgacggggccggtgaaggaaaagatcatggcggtgatcaaggagtggggcactggccgggacaccctgcgctgcttggccctggccacccgggacacccccccgaagcgagaggaaatggtcctggatgactctgccaggttcctggagtatgagacggacctgacattcgtgggtgtagtgggcatgctggaccctccgcgcaaggaggtcacgggctccatccagctgtgccgtgacgccgggatccgggtgatcatgatcactggggacaacaagggcacagccattgccatctgccggcgaattggcatctttggggagaacgaggaggtggccgatcgcgcctacacgggccgagagttcgacgacctgcccctggctgaacagcgggaagcctgccgacgtgcctgctgcttcgcccgtgtggagccctcgcacaagtccaagattgtggagtacctgcagtcctacgatgagatcacagccatgacaggtgatggcgtcaatgacgcccctgccctgaagaaggctgagattggcattgccatgggatctggcactgccgtggccaagactgcctctgagatggtgctggctgacgacaacttctccaccatcgtagctgctgtggaggagggccgcgccatctacaacaacatgaagcagttcatccgctacctcatttcctccaacgtgggcgaggtggtctgtatcttcctgaccgctgccctggggctgcctgaggccctgatcccggtgcagctgctatgggtgaacttggtgaccgacgggctcccagccacagccctgggcttcaacccaccagacctggacatcatggaccgccccccccggagccccaaggagcccctcatcagtggctggctcttcttccgctacatggcaatcgggggctatgtgggtgcagccaccgtgggagcagctgcctggtggttcctgtacgctgaggatgggcctcatgtcaactacagccagctgactcacttcatgcagtgcaccgaggacaacacccactttgagggcatagactgtgaggtcttcgaggcccccgagcccatgaccatggccctgtccgtgctggtgaccatcgagatgtgcaatgcactgaacagcctgtccgagaaccagtccctgctgcggatgccaccctgggtgaacatctggctgctgggctccatctgcctctccatgtccctgcacttcctcatcctctatgttgaccccctgccgatgatcttcaagctccgggccctggacctcacccagtggctcatggtcctcaagatctcactgccagtcattgggctcgacgaaatcctcaagttcgttgctcggaactacctagagggataactgttccccctcctccatctctgagcccgtgtcacagatccagaagatgaaagaaggaagtgagcatccttttgctctgtcctccccaccccgatagtgacacatcttcaggcagagctgtggcacagacccccgtcctgtcccccacacccgtgtcatgtgtctgttttataaacatgtccccttccctttccttccccctcggccacccgcctccctctcaaccttgtaaattccccttcccaaccccgaggggcttgcagggacaaggcgaccgactgcgctgagctgcttatttattgaaaataaacgacggaaaagtca

SEQ ID NO: 28 - protein, 417 aa, Homo sapiens
MEAAHAKTTEECLAYFGVSETTGLTPDQVKRNLEKYGLNELPAEEGKTLWELVIEQFEDLLVRILLLAACISFVLAWFEEGEETITAFVEPFVILLILIANAIVGVWQERNAENAIEALKEYEPEMGKVYRADRKSVQRIKARDIVPGDIVEVAVGDKVPADIRILAIKSTTLRVDQSILTGESVSVIKHTEPVPDPRAVNQDKKNMLFSGTNIAAGKALGIVATTGVGTEIGKIRDQMAATEQDKTPLQQKLDEFGEQLSKVISLICVAVWLINIGHFNDPVHGGSWFRGAIYYFKIAVALAVAAIPEGLPAVITTCLALGTRRMAKKNAIVRSLPSVETLGCTSVICSDKTGTLTTNQMSVCKMFIIDKVDGDICLLNEFSITGSTYAPEGEVPKVSMRRSARPPRQHSPPWWRR

SEQ ID NO: 29 - DNA, 3101 bp, Homo sapiens
  CDS (1)..(1146)
atggaggccgctcatgctaaaaccacggaggaatgtttggcctattttggggtgagtgagaccacgggcctcaccccggaccaagttaagcggaatctggagaaatacggcctcaatgagctccctgctgaggaagggaagaccctgtgggagctggtgatagagcagtttgaagacctcctggtgcggattctcctcctggccgcatgcatttccttcgagcggaacgcagagaacgccatcgaggccctgaaggagtatgagccagagatggggaaggtctaccgggctgaccgcaagtcagtgcaaaggatcaaggctcgggacatcgtccctggggacatcgtggaggtggctgtgggggacaaagtccctgcagacatccgaatcctcgccatcaaatccaccacgctgcgggttgaccagtccatcctgacaggcgagtctgtatctgtcatcaaacacacggagcccgttcctgacccccgagctgtcaaccaggacaagaagaacatgcttttctcgggcaccaacattgcagccggcaaggccttgggcatcgtggccaccaccggtgtgggcaccgagattgggaagatccgagaccaaatggctgccacagaacaggacaagacccccttgcagcagaagctggatgagtttggggagcagctctccaaggtcatctccctcatctgtgtggctgtctggcttatcaacattggccacttcaacgaccccgtccatgggggctcctggttccgcggggccatctactactttaagattgccgtggccttggctgtggctgccatccccgaaggtcttcctgcagtcatcaccacctgcctggccctgggtacccgtcggatggcaaagaagaatgccattgtaagaagcttgccctccgtagagaccctgggctgcacctctgtcatctgttccgacaagacaggcaccctcaccaccaaccagatgtctgtctgcaagatgtttatcattgacaaggtggatggggacatctgcctcctgaatgagttctccatcaccggctccacttacgctccagagggagaggtgccaaaggtgtctatgagaaggtcggcgaggccaccgagacagcactcaccaccctggtggagaagatgaatgtgttcaacacggatgtgagaagcctctcgaaggtggagagagccaacgcctgcaactcggtgatccgccagctaatgaagaaggaattcaccctggagttctcccgagacagaaagtccatgtctgtctattgctccccagccaaatcttcccgggctgctgtgggcaacaagatgtttgtcaagggtgcccctgagggcgtcatcgaccgctgtaactatgtgcgagttggcaccacccgggtgccactgacggggccggtgaaggaaaagatcatggcggtgatcaaggagtggggcactggccgggacaccctgcgctgcttggccctggccacccgggacacccccccgaagcgagaggaaatggtcctggatgactctgccaggttcctggagtatgagacggacctgacattcgtgggtgtagtgggcatgctggaccctccgcgcaaggaggtcacgggctccatccagctgtgccgtgacgccgggatccgggtgatcatgatcactggggacaacaagggcacagccattgccatctgccggcgaattggcatctttggggagaacgaggaggtggccgatcgcgcctacacgggccgagagttcgacgacctgcccctggctgaacagcgggaagcctgccgacgtgcctgctgcttcgcccgtgtggagccctcgcacaagtccaagattgtggagtacctgcagtcctacgatgagatcacagccatgacaggtgatggcgtcaatgacgcccctgccctgaagaaggctgagattggcattgccatgggatctggcactgccgtggccaagactgcctctgagatggtgctggctgacgacaacttctccaccatcgtagctgctgtggaggagggccgcgccatctacaacaacatgaagcagttcatccgctacctcatttcctccaacgtgggcgaggtggtctgtatcttcctgaccgctgccctggggctgcctgaggccctgatcccggtgcagctgctatgggtgaacttggtgaccgacgggctcccagccacagccctgggcttcaacccaccagacctggacatcatggaccgccccccccggagccccaaggagcccctcatcagtggctggctcttcttccgctacatggcaatcgggggctatgtgggtgcagccaccgtgggagcagctgcctggtggttcctgtacgctgaggatgggcctcatgtcaactacagccagctgactcacttcatgcagtgcaccgaggacaacacccactttgagggcatagactgtgaggtcttcgaggcccccgagcccatgaccatggccctgtccgtgctggtgaccatcgagatgtgcaatgcactgaacagcctgtccgagaaccagtccctgctgcggatgccaccctgggtgaacatctggctgctgggctccatctgcctctccatgtccctgcacttcctcatcctctatgttgaccccctgccgatgatcttcaagctccgggccctggacctcacccagtggctcatggtcctcaagatctcactgccagtcattgggctcgacgaaatcctcaagttcgttgctcggaactacctagagggataactgttccccctcctccatctctgagcccgtgtcacagatccagaagatgaaagaaggaagtgagcatccttttgctctgtcctccccaccccgatagtgacacatcttcaggcagagctgtggcacagacccccgtcctgtcccccacacccgtgtcatgtgtctgttttataaacatgtccccttccctttccttccccctcggccacccgcctccctctcaaccttgtaaattccccttcccaaccccgaggggcttgcagggacaaggcgaccgactgcgctgagctgcttatttattgaaaataaacgacggaaaagtca

SEQ ID NO: 30 - protein, 382 aa, Homo sapiens
MEAAHAKTTEECLAYFGVSETTGLTPDQVKRNLEKYGLNELPAEEGKTLWELVIEQFEDLLVRILLLAACISFERNAENAIEALKEYEPEMGKVYRADRKSVQRIKARDIVPGDIVEVAVGDKVPADIRILAIKSTTLRVDQSILTGESVSVIKHTEPVPDPRAVNQDKKNMLFSGTNIAAGKALGIVATTGVGTEIGKIRDQMAATEQDKTPLQQKLDEFGEQLSKVISLICVAVWLINIGHFNDPVHGGSWFRGAIYYFKIAVALAVAAIPEGLPAVITTCLALGTRRMAKKNAIVRSLPSVETLGCTSVICSDKTGTLTTNQMSVCKMFIIDKVDGDICLLNEFSITGSTYAPEGEVPKVSMRRSARPPRQHSPPWWRR